(19) United States
(12) Patent Application Publication
(10) Pub. No.: US 2012/0004201 A1
(43) Pub. Date: Jan. 5, 2012

(54) PHOSPHODIESTARASE INHIBITORS

(76) Inventors: Sonali Rudra, Salt Lake (IN); Nidhi Gupta, New Delhi (IN); Kakade Ganesh Chandrakant, Ahmednagar (IN); Tarun Jain, Ghaziabad (IN); Sreedhara Rao Voleti, Hyderabad (IN); Abhijit Ray, New Delhi (IN); Sunanda G. Dastidar, New Delhi (IN); Lalitha Vijaykrishnan, New Delhi (IN)

(21) Appl. No.: 13/060,832

(22) PCT Filed: Sep. 19, 2009
(86) PCT No.: PCT/IB09/54115

§ 371 Date: Sep. 22, 2011

(30) Foreign Application Priority Data
Sep. 19, 2008 (IN) 2005/DEL/2008

(51) Int. Cl. A61K 31/56; C07D 519/00; A61K 31/437; A61P 29/00; A61P 25/00; A61P 37/06; A61P 37/00; A61P 11/00; A61P 11/06; A61P 11/08; A61P 11/02; A61P 17/06; A61P 17/00; A61P 43/00; A61P 27/02; A61P 19/02; A61P 1/00; A61P 35/00; A61P 1/18; C07D 471/04

(52) U.S. Cl. 514/171; 546/15; 546/119; 514/278; 514/303

(57) ABSTRACT

The present invention relates to phosphodiesterase (PDE) type 4, phosphodiesterase (PDE) type 7 and dual PDE type 4/PDE type 7 inhibitors. Compounds disclosed herein can be useful in the treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans. Processes for the preparation of disclosed compounds, pharmaceutical compositions containing the disclosed compounds and their use as PDE type 4, PDE type 7 and dual PDE type 4/PDE type 7 inhibitors are provided.

### FIELD OF THE INVENTION

The present invention relates to phosphodiesterase (PDE) type 4, phosphodiesterase (PDE) type 7 and dual PDE type 4/PDE type 7 inhibitors.

Compounds disclosed herein can be useful in the treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Processes for the preparation of disclosed compounds, pharmaceutical compositions containing the disclosed compounds and their use as PDE type 4, PDE type 7 and dual PDE type 4/PDE type 7 inhibitors are provided.

### BACKGROUND OF THE INVENTION

It is known that cyclic adenosine-3′, 5′-monophosphate (cAMP) exhibits an important role of acting as an intracellular secondary messenger (Pharmacol. Rev., 12, (1960), 265). Its intracellular hydrolysis to adenosine 5′-monophosphate (AMP) causes number of inflammatory conditions which are not limited to COPD, asthma, arthritis, psoriasis, allergic rhinitis, shock, atopic dermatitis, Crohn's disease, adult respiratory distress syndrome (ARDS), eosinophilic granuloma, allergic conjunctivitis, osteoarthritis or colitis. PDE4 inhibitors are designed to inhibit the activity of PDE4, the enzyme which breaks down neuronal cAMP. Studies have shown that administering PDE4 inhibitors can have a restorative effect on memory loss in animal models, including those of Alzheimer's disease (Expert Opin. Ther. Targ., 9 (6): (2005) 1283-1305; Drug Discovery Today, 10, 22: (2005), 1503-1519). The most important role in the control of cAMP (as well as of cGMP (cyclic guanosine monophosphate)) level is played by cyclic nucleotide phosphodiesterases (PDE) which represent a biochemically and functionally highly variable super family of enzymes. Eleven distinct families of cyclic nucleotide phosphodiesterases with more than 25 gene products are currently recognized Although PDE1, PDE2, PDE3, PDE4, and PDE7 all use cAMP as a substrate, only PDE4 and PDE7 are highly selective for hydrolysis of cAMP. Inhibitors of PDE, particularly the PDE4 inhibitors, such as rolipram or Ro-1724 are therefore known as cAMP-enhancers. Immune cells contain type 4 and type 3 PDE, the PDE4 type being prevalent in human mononuclear cells. Thus the inhibition of phosphodiesterase type 4 has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. Distinct classes of PDE's have been recognized (TIPS, 11, (1990), 150), and their selective inhibition has led to improved drug therapy (TIPS, 12, (1991), 19). Thus it was recognized that inhibition of PDE4 could lead to inhibition of inflammatory mediator release (J. Mol. Cell. Cardiol., 12 (Suppl. II), (1989), S 61) and airway smooth muscle relaxation.

The current approach of targeting PDE4 for alleviating the chronic inflammation associated with COPD is compromised by the dose limiting side effects that are proving difficult to overcome. Theoretically, an alternate strategy would be to use small molecule inhibitors to target other members of the cAMP dependent PDE family that share a common pulmonary cellular distribution to PDE4. It is hypothesized that such an approach would yield compounds with an improved therapeutic ratio. Of the novel cAMP family of proteins discovered so far, PDE7A offers itself as a promising candidate because of its cellular distribution in almost all pro inflammatory and immune cells (Curr Pharm Des., 12, (2006), 1-14). Additionally, it has been shown to be a prime modulator of human T cell function as well (Science; 283 (5403): Feb 5, (1999), 848-851).

Thus, dual specificity inhibitors that target both PDE4 and PDE7 would in principle, have an improved spectrum and a wider therapeutic window in the clinics. Compounds with dual PDE4 and PDE7 inhibitory effects have been shown to inhibit T cell function such as cytokine production, proliferation and activation of CD25 expression markers on T cells induced by antigen stimulation (Eur. J. Pharmacol., 541, (2006), 106-114). Development of dual PDE4-PDE7 inhibitors would yield a novel class of drugs blocking T cell component of a disease partly through PDE7 inhibition as well as possess anti-inflammatory activity. (Eur. J. Pharmacol., 550, (2006), 166-172 Eur. J. Pharmacol., 559, (2007), 219-226). More importantly, such a pharmacophore would be less limited by nausea and vomiting, a major side effect associated with PDE4 inhibition.

WO 03/047520 discloses substituted aminomethyl compounds and derivatives thereof, which have been described to be useful as inhibitors of factor Xa. WO 00/59902 discloses aryl sulfonyls, which have been described to be useful as inhibitors of factor Xa. WO 97/48697 discloses substituted azabicyclic compounds and their use as inhibitors of the production of TNF and cyclic AMP phosphodiesterase. WO 98/57951 and U.S. Pat. No. 6,339,099 describe nitrogen containing heteroaromatics and derivatives, which have been said to be the inhibitors of factor Xa. WO 2005/063767 and WO 2006/001894 disclose indoles, 1H-indazoles, 1,2-benzisoxazoles, and 1,2-benzisothiazoles, preparation and uses thereof WO 2007/031838, WO 2007/031977, WO 2008/111009 and WO 2008/111010 discloses substituted pyrazolo [3,4-b]pyridines as phosphodiesterase inhibitors.

### SUMMARY OF THE INVENTION

The present invention provides phosphodiesterase (PDE) type 4, PDE type 7 and dual PDE type 4/PDE type 7 inhibitors, which can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Pharmaceutically acceptable salts, pharmaceutically acceptable solvates, stereoisomers, tautomers, racemates, regioisomers, geometric isomers, prodrugs, metabolites, polymorphs or N-oxides of these compounds having the same type of activity are also provided.

Pharmaceutical compositions containing the compounds, which may also contain pharmaceutically acceptable carriers, excipients or diluents, can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

Other aspects will be set forth in the accompanying description which follows and in part will be apparent from the description or may be learnt by the practice of the invention.

In accordance with one aspect, there are provided compounds having the structure of Formula I:

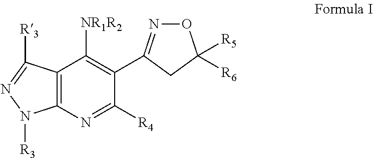

or their pharmaceutically acceptable salts, pharmaceutically acceptable solvates, stereoisomers, tautomers, racemates, regioisomers, geometric isomers, prodrugs, metabolites, polymorphs or N-oxides, wherein R₁ and R₂ independently can be hydrogen, aryl, heteroaryl, —COR₇, —S(O)ₘR₇ (wherein R₇ can be hydrogen, alkyl, cycloalkyl, aryl,

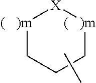

aralkyl, heteroaryl or heterocyclyl and m can be an integer from 0-2), or (wherein m can be an integer from 0-2 and X can be —O—, S(O)ₘ (wherein m can be an integer from 0-2), NR₈ {wherein R₈ can be hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ (wherein R₇ and R′₇ can be hydrogen, alkyl, cycloalkyl, aryl, aralkyl, heteroaryl or heterocyclyl and m can be the same as defined earlier)}, C(═O), C═NOH or CR_{f}R_{q} (wherein R_{f} and R_{q} independently can be hydrogen, halogen, hydroxy, cyano, NR₈R′₈ [wherein R₈ and R′₈ can be hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ {wherein m, R₇ and R′₇ can be the same as defined earlier}], —CONR₇R₇′, —COONR₇R₇′ or —COOR₇ (wherein R₇ and R′₇ can be the same as defined earlier)),

R₃ can be alkyl, aryl, cycloalkyl, heterocyclyl or heteroaryl,

R′₃ can be hydrogen, alkyl, aryl, cycloalkyl, heteroaryl, heterocyclyl or (un) substituted amine,

R₄ can be alkyl, aryl, cycloalkyl, halogen, cyano, heteroaryl, heterocyclyl, or (un) substituted amine,

R₅ and R₆ independently can be alkyl, —CN, heterocyclyl, —(CH₂)ₘC(═O)NRⱼR′ⱼ {wherein m can be an integer from 0-2 and Rⱼ and R′ⱼ independently can be hydrogen, alkyl, alkenyl, cycloalkyl, aryl, aralkyl, heterocyclyl, heteroaryl, heterocyclylalkyl, heteroarylalkyl or Rⱼ and R′ⱼ taken together with the nitrogen atom to which they are attached can form a optionally substituted heterocyclyl ring}, —(CH₂)ₘC(═O)ORⱼ {wherein m and Rⱼ can be the same as defined earlier}, —(CH₂)ₘₗORⱼ {wherein ml can be an integer from 0-3 and Rⱼ can be the same as defined earlier}or —(CH₂)ₘC(═O)heterocyclyl {wherein m can be the same as defined earlier}, or R₅ and R₆ together can form a 3-7 membered saturated, partially saturated or unsaturated ring containing carbon atoms wherein one or more carbon atoms optionally can be replaced by heteroatoms selected from O, S(O)ₘ {wherein m can be an integer from 0-2}or NR₈ {wherein R₈ can be the same as defined earlier}, or one or more carbon atoms optionally can be substituted with oxo, spiro-attached heterocyclyl, cyano, alkyl, heteroaryl, heteroarylalkyl, —(CH₂)ₘhalogen, —(CH₂)ₘNR₇R′₇, —(CH₂)ₘOR₇, —(CH₂)ₘCONR₇R′₇, —(CH₂)ₘNR₇COR₇ or —(CH₂)ₘCOOR₇ (wherein m, R₇ and R′₇ can be the same as defined earlier).

In one embodiment, there are provided compounds having the structure of Formula II:

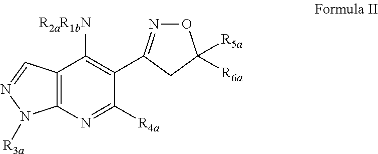

or their pharmaceutically acceptable salts, pharmaceutically acceptable solvates, stereoisomers, tautomers, racemates, regioisomers, geometric isomers, prodrugs, metabolites, polymorphs or N-oxides, wherein R_{1b} and R₂ₐ independently can be

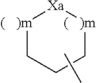

hydrogen, or (wherein m can be an integer from 0-2 and Xa can be —O— or —CH₂—),

R₃ₐ can be alkyl,

R₄ₐ can be alkyl, which may optionally be substituted with halogen,

R₅ₐ and R₆ₐ independently can be —(_{CH}₂)ₘ₁OH {wherein m1 can be an integer from 0-3}or R₅ₐ and R₆ₐ together can form a 3-7 membered saturated, partially saturated or unsaturated ring containing carbon atoms wherein one or more carbon atoms optionally can be replaced by heteroatoms selected from O, S(O)ₘ{wherein m can be an integer from 0-2}or NR₈ {wherein R₈ can be hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ (wherein R₇ and R′₇ can be hydrogen, alkyl, cycloalkyl, aryl, aralkyl, heteroaryl or heterocyclyl and m can be the same as defined earlier)}, or one or more carbon atoms optionally can be substituted with oxo, spino-attached heterocyclyl, cyano, heteroaryl, heteroarylalkyl, —(CH₂)ₘhalogen, —(CH₂)ₘOR₇, or —(CH₂)ₘCOOR₇ (wherein R₇ and m can be the same as defined earlier).

The following definitions apply to terms as used herein.

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, or aryl groups,

The term “alkenyl,” unless otherwise specified, refers to a monoradical of a branched or unbranched unsaturated hydrocarbon group having from 2 to 20 carbon atoms with cis, trans or geminal geometry. Alkenyl groups may be substituted further with one or more substituents selected from alkyl, alkenyl, alkynyl, cycloalkyl, cycloalkenyl, aryl, aralkyl, heterocyclyl, heteroaryl, heterocyclylalkyl, or heteroarylalkyl groups

The term “alkynyl,” unless otherwise specified, refers to a monoradical of an unsaturated hydrocarbon, having from 2 to 20 carbon atoms. Alkynyl groups may be substituted further with one or more substituents selected from alkyl, alkenyl, alkoxy, cycloalkyl, cycloalkenyl, aryl, or aralkyl groups.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentyl, cyclohexyl and the like or multiple ring structures, including adamantanyl, and bicyclo [2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, aryl, or aralkyl groups.

The term “cycloalkylalkyl” refers to alkyl-cycloalkyl group linked through alkyl portion, wherein the alkyl and cycloalkyl are as defined earlier.

The term “alkoxy” denotes the group O-alkyl wherein alkyl is the same as defined above.

The term “aryl,” unless otherwise specified, refers to aromatic system having 6 to 14 carbon atoms, wherein the ring system can be mono-, bi- or tricyclic and carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, —COOR_{ψ}, —NHC(═O)R_{λ}, R_{π}, —C(═O)NR_{λ},R_{π}, —NHC(=O)NR_{λ}R_{π}, —O—C(═O)NR_{λ}R_{π}, —SOₘR_{ψ}, carboxy, heterocyclyl, heteroaryl, heterocyclylalkyl, heteroarylalkyl, amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π}, or —SO₂NHR_{π}(wherein m is an integer from 0-2; R_{λ}, R_{π} and R_{ψ} are independently selected from hydrogen, halogen, hydroxy, alkyl, alkenyl, alkynyl, alkoxy, cycloalkyl, cycloalkenyl, aryl, aralkyl, heterocyclyl, heteroaryl, heterocyclylalkyl, heteroarylalkyl or carboxy). Aryl groups optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl and aryl are as defined above). Examples of aralkyl groups include benzyl, ethylphenyl, propylphenyl, naphthylmethyl and the like.

The term “aralkenyl,” unless otherwise specified, refers to alkenyl-aryl linked through alkenyl portion (wherein alkenyl and aryl are as defined above).

The term “aryloxy” denotes the group O-aryl, wherein aryl is as defined above.

The term “cycloalkoxy” denotes the group O-cycloalkyl, wherein cycloalkyl is as defined above.

The term “carboxy,” as defined herein, refers to —C(═O)OR_{ψ}, wherein R_{ψ}, is the same as defined above.

The term “heteroaryl,” unless otherwise specified, refers to a monocyclic aromatic ring structure containing 5 or 6 ring atoms or a bicyclic or tricyclic aromatic group having from 8 to 10 ring atoms, with one or more heteroatom(s) independently selected from N, O or S and optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, —CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ},R_{π}—NHC(═O)NR_{λ}R_{π}, —SOₘR_{ψ}, —O—C(═O)NR_{λ}R_{π}, —O—C(═O)R_{λ}, or —O—C(═O)OR_{λ}, (wherein m, R_{ψ}, R_{λ}, and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic cycloalkyl group having 5 to 10 atoms wherein 1 to 4 carbon atoms in a ring are replaced by heteroatoms selected from O, S(O)ₘ (wherein m is an integer from 0-2) or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, —SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ, —NHSO}₂R_{λ}, or SO₂NHR_{λ} (wherein m, R_{ψ}, R_{λ}, and R_{π} are as defined earlier) or guanidine. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include tetrahydropyranyl, oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, isoindole 1,3-dione, piperidinyl, piperazinyl, 3H-imidazo[4,5-b]pyridine, isoquinolinyl, dioxolanyl, 1H-pyrrolo[2,3-b]pyridine or piperazinyl and the like.

“Spiro-attached heterocyclyl” refers to heterocyclyl group attached to ring formed by R₅ and R₆ of Formula I or R₅ₐ and R₆ₐ of Formula II via one carbon atom common to both rings, i.e. ring formed by R₅ and R₆ or R₅ₐ and R₆ₐ and heterocyclyl ring.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R_{z} (wherein R_{z} is alkyl, cycloalkyl, aryl, aralkyl, heteroaryl, heterocyclyl, heteroarylalkyl or heterocyclylalkyl).

“Amine,” unless otherwise specified, refers to —NH₂. “Substituted amine” unless otherwise specified, refers to a group —N(Rₖ)₂ wherein each Rₖ is independently selected from the group hydrogen provided that both Rₖ groups are not hydrogen (defined as “amine”), alkyl, alkenyl, alkynyl, aralkyl, cycloalkyl, aryl, heteroaryl, heterocyclyl, heterocyclylalkyl, or heteroarylalkyl groups.

“Thiocarbonyl” refers to —C(═S)H. Thiocarbonyl may be substituted and “substituted thiocarbonyl” refers to —C (═S) R″', wherein R′″ is selected from alkyl, cycloalkyl, aryl, aralkyl, heteroaryl, heterocyclyl, heteroarylalkyl, heterocyclylalkyl, amine or substituted amine.

The term “oxo” means “═O”. Oxo is attached at a carbon atom unless otherwise noted. Oxo, together with the carbon atom to which it is attached forms a carbonyl group (i.e., C═O).

The term “halogen” refers to fluorine, chlorine, bromine or iodine.

The compounds of the present invention can be used for treatment, prevention, inhibition or suppression of CNS diseases, for example, multiple sclerosis; various pathological conditions such as diseases affecting the immune system, including AIDS, rejection of transplant, auto-immune disorders such as T-cell related diseases, for example, rheumatoid arthritis; inflammatory diseases such as respiratory inflammation diseases including chronic obstructive pulmonary disease (COPD), asthma, bronchitis, allergic rhinitis, adult respiratory distress syndrome (ARDS) and other inflammatory diseases including but not limited to psoriasis, shock, atopic dermatitis, eosinophilic granuloma, allergic conjunctivitis, osteoarthritis; gastrointestinal inflammation diseases such as Crohn's disease, colitis, pancreatitis as well as different types of cancers including leukaemia; especially in humans.

In accordance with yet another aspect, there are provided processes for the preparation of the compounds as described herein.

### DETAILED DESCRIPTION OF THE INVENTION

The compounds described herein may be prepared by techniques well known in the art and familiar to the average synthetic organic chemist. In addition, the compounds of present invention may be prepared by the following, for example, reaction sequences as depicted in Schemes I, II, III IV and V.

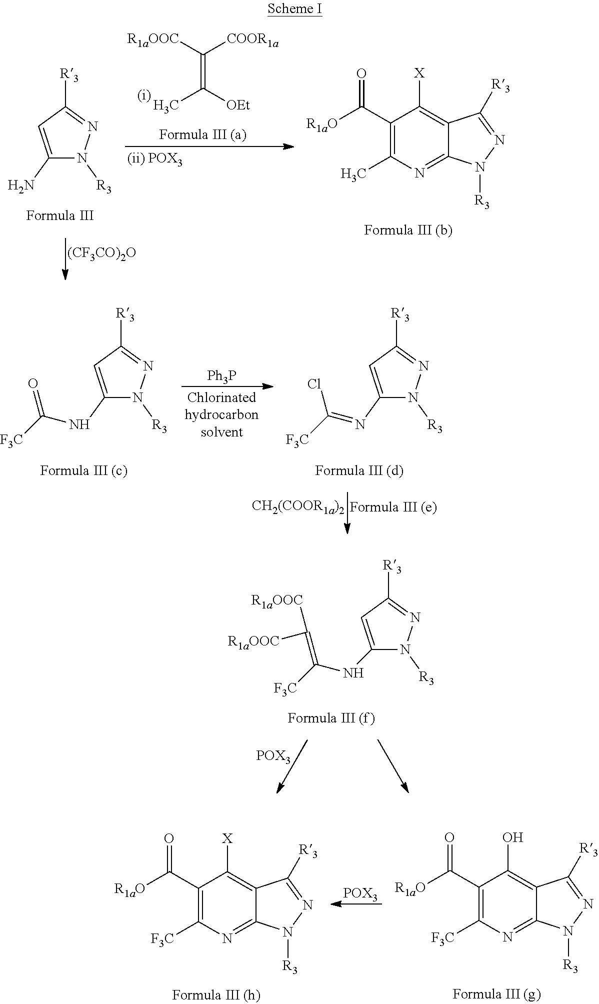

The compounds of Formula III (b) and III (h) can be prepared by following Scheme I. Accordingly, compounds of Formula III are reacted with

(a) compounds of Formula III (a) followed by the reaction with phosphorous oxy halide to give compounds of Formula III (b) (wherein R₁ₐ is alkyl , X is a halogen and R₃ and R′₃ are the same as defined earlier).

(b) trifluoro acetic anhydride to give compounds of Formula III (c), which are reacted with triphenylphosphine to give compounds of Formula III(d) , which are reacted with compounds of Formula III (e) to give compounds of Formula III (f), which
(i) on reaction with phosphorous oxy halide give compounds of Formula III (h) (wherein R₁ₐ, X, R₃ and R′₃ are the same as defined earlier).
(ii) on refluxing gives compounds of Formula III (g), which on reaction with phosphorous oxy halide give compounds of Formula III (h) (wherein R₁ₐ, X, R₃ and R′₃ are the same as defined earlier).

The compounds of Formula III (b) can be prepared by the reaction of compounds of Formula III with compounds of Formula III (a) on heating followed by the reaction with phosphorous oxy halide.

The reaction of compounds of Formula III with trifluoro acetic anhydride to give compounds of Formula III (c) can be carried out in one or more solvents, for example, nitriles, for example, acetonitrile; ketones, for example, acetone; ethers, for example, diethyl ether or tetrahydrofuran; amides, for example, dimethylfoimamide or dimethylacetamide; sulfoxides, for example, dimethylsulfoxide; hydrocarbons, for example, hexane or toluene; halogenated hydrocarbons, for example, dichloromethane, dichloroethane, chloroform or carbon tetrachloride; or mixture(s) thereof.

The reaction of compounds of Formula III (c) with triphenylphosphine to give compounds of Formula III(d) can be carried out in one or more chlorinated hydrocarbon solvents, for example, dichloromethane, dichloroethane, chloroform or carbon tetrachloride; or mixture(s) thereof.

The reaction of compounds of Formula III(d) with compounds of Formula III (e) to give compounds of Formula III (f) can be carried out in the presence of one or more bases, for example, triethylamine, pyridine, potassium tert- butoxide, sodium hydride or mixture(s) thereof.

The reaction of compounds of Formula III(d) with compounds of Formula III (e) to give compounds of Formula III (f) can be carried out in one or more solvents, for example, nitriles, for example, acetonitrile; ethers, for example, diethyl ether or tetrahydrofuran; amides, for example, dimethylformamide or dimethylacetamide; sulfoxides, for example, dimethylsulfoxide; hydrocarbons, for example, hexane or toluene or mixture(s) thereof.

The compounds of Formula III (f) on refluxing in one or more solvents, for example, nitriles, for example, acetonitrile; ketones, for example, acetone; alcohols, for example, methanol, ethanol, propanol or butanol; ethers, for example, tetrahydrofuran; amides, for example, dimethylformamide or dimethylacetamide; sulfoxides, for example, dimethylsulfoxide; hydrocarbons, for example toluene; dipolar aprotic solvents, for example, N-methylpyrrolidone; or mixture(s) thereof can give compounds of Formula III (g).

The compounds of Formula III (g) on heating with phosphorous oxy halide can give compounds of Formula III (h).

Alternatively, the compounds of Formula III (f) on heating with phosphorous oxy halide can give compounds of Formula III (h).

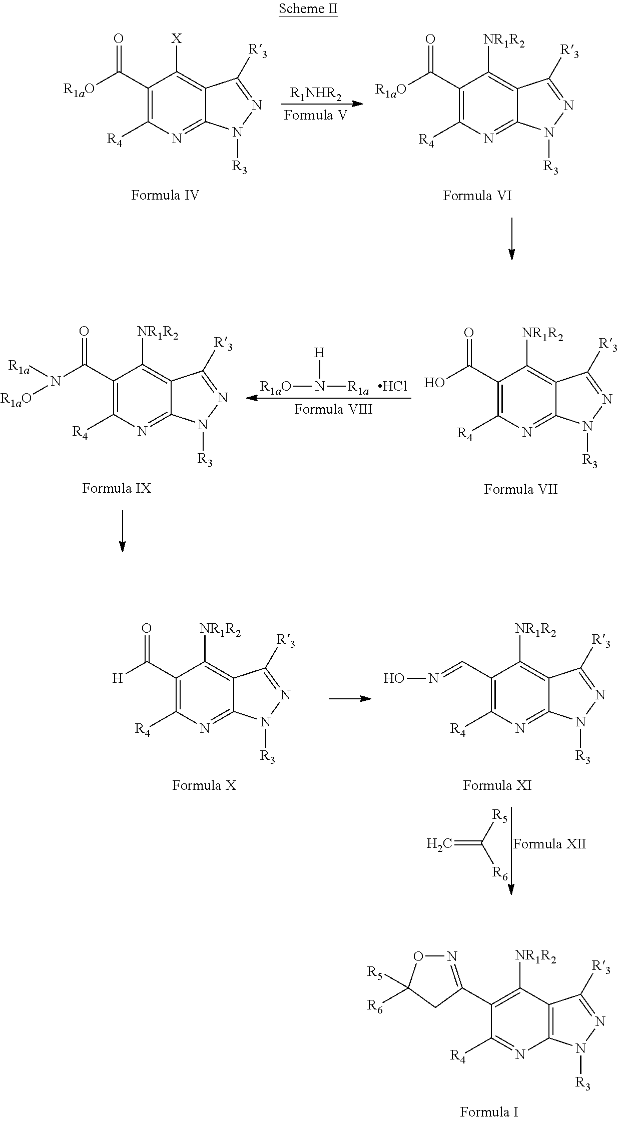

The compounds of Formula I can be prepared by following Scheme IL Accordingly, compounds of Formula IV (represents Formula III (b), when R₄ is —CH₃ and Formula III (h) when R₄ is —CF₃) are reacted with compounds of Formula V to give compounds of Formula VI, which on ester hydrolysis give compounds of Formula VII, which are reacted with compounds of Formula VIII (wherein R₁ₐ is the same as defined earlier) to give compounds of Formula IX, which on reduction give compounds of Formula X, which on reaction with hydroxylamine hydrochloride give compounds of Formula XI, which are finally reacted with compounds of Formula XII to give compounds of Formula I (wherein R₁, R₂, R₃, R′₃, R₄, R₅ and R₆ are the same as defined earlier).

The reaction of compounds of Formula IV with compounds of Formula V to give compounds of Formula VI can be carried out in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; dipolar aprotic solvents, for example, N-methylpyrrolidone; or mixture(s) thereof.

The reaction of compounds of Formula IV with compounds of Formula V can be carried out in the optional presence of one or more bases, for example, triethylamine, pyridine, potassium tent- butoxide, sodium hydride or mixture(s) thereof.

The ester hydrolysis of compounds of Formula VI to give compounds of Formula VII can be carried out in one or more solvents, for example, alcohols, for example, methanol, ethanol, propanol or butanol; or an alcohol and water mixture.

The ester hydrolysis of compounds of Formula VI to give compounds of Formula VII can be carried out in the presence of one or more inorganic bases, for example, alkali metal hydroxides, for example, potassium hydroxide, sodium hydroxide, lithium hydroxide or mixture(s) thereof.

The reaction of compounds of Formula VII with compounds of Formula VIII to give compounds of Formula IX can be carried out in the presence of one or more activating reagents, for example, hydroxybenzotriazole, acetone oxime, 2-hydroxypyridine or mixture(s) thereof, and one or more coupling reagents, for example, 1-ethyl-3-(3-dimethylaminopropyl) carbodiimide hydrochloride, 1,3-dicyclohexyl carbodiimide or mixture(s) thereof in one or more solvents, for example, ethers, for example, diethyl ether or tetrahydrofuran; amides, for example, dimethylformamide or dimethylacetamide; sulfoxides, for example, dimethylsulfoxide; or mixture(s) thereof.

The reaction of compounds of Formula VII with compounds of Formula VIII can be carried out in the presence of one or more bases, for example, N-methylmorpholine; N-ethyldiisopropylamine; 4-dialkylaminopyridines, for example, 4-dimethylaminopyridine; or mixture(s) thereof.

The reduction of compounds of Formula IX to give compounds of Formula X can be carried out in one or more solvents, for example, ethers, for example, diethyl ether or tetrahydrofuran; amides, for example, dimethylformamide or dimethylacetamide; sulfoxides, for example, dimethylsulfoxide; hydrocarbons, for example, hexane or toluene; or mixture(s) thereof.

The reduction of compounds of Formula IX to give compounds of Formula X can be carried out in the presence of one or more reducing agents, for example, sodium his (2-methoxyethoxy)aluminum hydride (vitride), lithium aluminium hydride or mixture(s) thereof.

The reaction of compounds of Formula X with hydroxylamine hydrochloride to give compounds of Formula XI can be carried out in the presence of sodium acetate in one or more solvents, for example, alcohols, for example, methanol, ethanol, propanol, butanol or mixture(s) thereof.

The reaction of compounds of Formula XI with compounds of Formula XII to give compounds of Formula I can be carried out in the presence of one or more halogenating agents, for example, sodium hypochlorite, N-chlorosuccinimide, N-bromosuccinimide or mixture(s) thereof, in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; halogenated hydrocarbons, for example, dichloromethane, dichloroethane or chloroform; or mixture(s) thereof.

The reaction of compounds of Formula XI with compounds of Formula XII can be carried out in the optional presence of one or more bases, for example, triethyl amine, trimethyl amine or mixture(s) thereof.

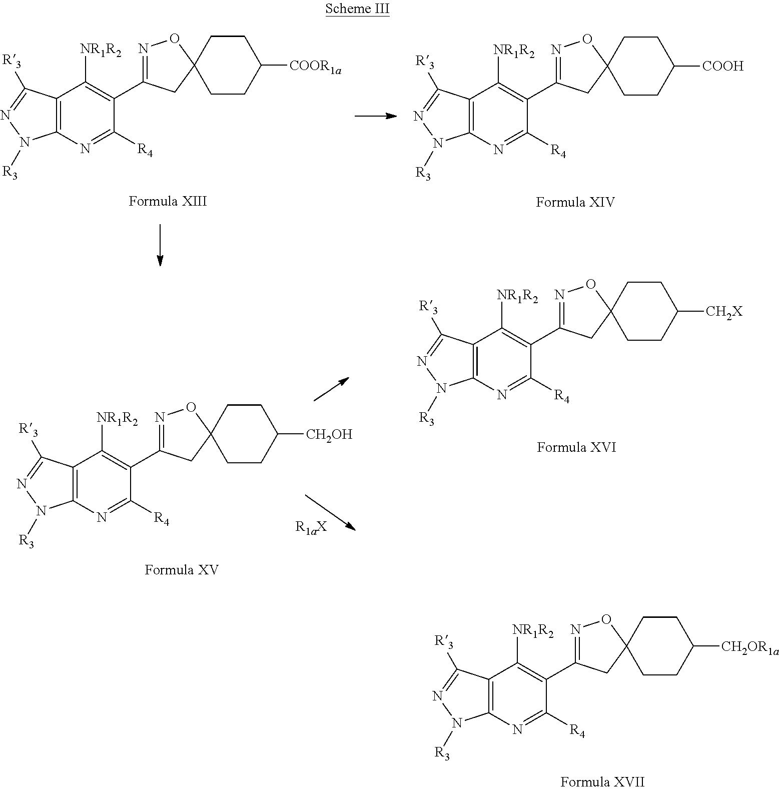

The compounds of Formulae XIV, XV, XVI and XVII can be prepared by following Scheme III. Accordingly, compounds of Formula XIII (wherein R₁ₐ is alkyl),

(i) on ester hydrolysis give compounds of Formula XIV (wherein R₁, R₂, R₃, R′₃ and R₄ are the same as defined earlier).

(ii) on reduction give compounds of Formula XV,
(a) which are halogenated to give compounds of Formula XVI (wherein

R₁, R₂, R₃, R′₃, R₄ are the same as defined earlier and X is halogen).
(b) which are reacted with compounds of Formula R₁ₐX (wherein X is halogen) to give compounds of Formula XVII (wherein R₁, R₂, R₃, R′₃, R₄ and R₁ₐ are the same as defined earlier).

The ester hydrolysis of compounds of Formula XIII to give compounds of Formula XIV can be carried out in one or more solvents, for example, water; ethers, for example, diethyl ether or tetrahydrofuran; alcohols, for example, methanol, ethanol, propanol or butanol; or mixture(s) thereof.

The ester hydrolysis of compounds of Formula XIII can be carried out in the presence of one or more inorganic bases, for example, alkali metal hydroxides, for example, potassium hydroxide, sodium hydroxide, lithium hydroxide or mixture(s) thereof.

The reduction of compounds of Formula XIII to give compounds of Formula XV can be carried out in the presence of one or more reducing agents, for example, sodium borohydride, lithium boro hydride, lithium aluminium hydride or hydrogen in the presence of palladium/carbon or mixture(s) thereof in one or more solvents, for example, ethers, for example, diethyl ether, dioxane or tetrahydrofuran; alcohols, for example, methanol, ethanol, propanol or butanol; or mixture(s) thereof.

The halogenation of compounds of Formula XV to give compounds of Formula XVI can be carried out with one or more halogenating agents, for example, diethylamino sulphurtrifluoride, thionyl chloride, phosphoryl chloride, thionyl bromide, phosphoryl bromide or mixture(s) thereof in one or more solvents, for example, nitriles, for example, acetonitrile; ketones, for example, acetone; alcohols, for example, methanol, ethanol, propanol or butanol; ethers, for example, diethyl ether or tetrahydrofuran; amides, for example, dimethylformamide or dimethylacetamide; sulfoxides, for example, dimethylsulfoxide; hydrocarbons, for example, hexane or toluene; halogenated hydrocarbons, for example, dichloromethane, dichloroethane or chloroform; or mixture(s) thereof. The reaction of compounds of Formula XV with compounds of Formula R₁ₐX to give compounds of Formula XVII can be carried out in one or more solvents, for example, amides, for example, dimethylformamide or dimethylacetamide; sulfoxides, for example, dimethylsulfoxide; or mixture(s) thereof.

The reaction of compounds of Formula XV with compounds of Formula R₁ₐX can be carried out in presence of one or more inorganic bases, for example, alkali metal hydrides, for example, potassium hydride or sodium hydride, alkali metal carbonates, for example, potassium carbonate, or mixture(s) thereof.

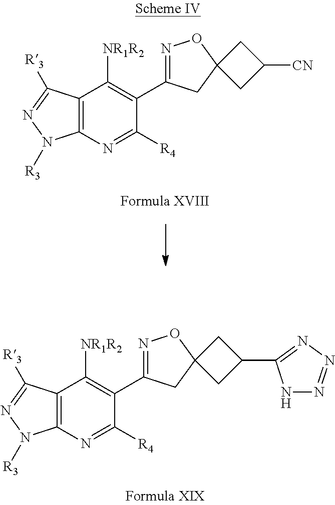

The compounds of Formula XIX can be prepared by following Scheme IV. Accordingly, compounds of Formula XVIII are cyclized to give compounds of Formula XIX (wherein R₁, R₂, R₃, R′₃ and R₄ are the same as defined earlier).

The cyclization of compounds of Formula XVIII to give compounds of Formula XIX can be carried out in the presence of sodium azide and triethyl amine hydrochloride in one or more solvents, for example, amides, for example, dimethylformamide or dimethylacetamide; sulfoxides, for example, dimethylsulfoxide; hydrocarbons, for example, hexane or toluene; or mixture(s) thereof.

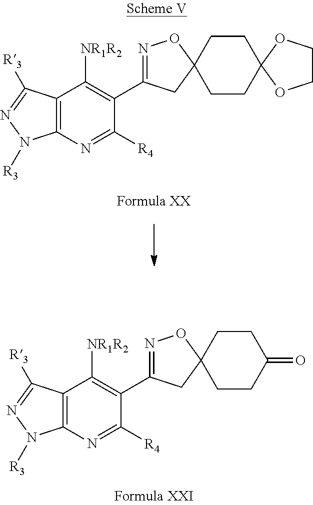

The compounds of Formula XXI can be prepared by following Scheme V. Accordingly, hydrolysis of compounds of Formula XX gives compounds of Formula XXI (wherein R₁, R₂, R₃, R′₃ and R₄ are the same as defined earlier).

The hydrolysis of compounds of Formula XX to give compounds of Formula XXI can be carried out in the presence of one or more acids, for example trifluoroacetic acid, p-toluene sulphonic acid or mixture(s) thereof in one or more solvents, for example, halogenated hydrocarbons, for example, dichloromethane, dichloroethane or chloroform; water or mixture(s) thereof.

Compounds of Formula II can also be prepared by following the Schemes I, II, III IV and V.

In the above schemes, where the specific solvents, bases, reducing agents, activating reagents, coupling reagents, acids, halogenating agents, etc., are mentioned, it is to be understood that other solvents, bases, reducing agents, activating reagents, coupling reagents, acids, halogenating agents, etc., known to those skilled in the art may be used. Similarly, reaction parameters such as the reaction temperature and duration may be adjusted according to the desired needs.

An illustrative list of compounds of the invention includes these listed below:

N-cyclohexyl-1-ethyl-6-methyl-5-[(cis*)-2-(1H-tetrazol-5-yl)-5-oxa-6-azaspiro[3.4]oct-6-en-7-yl]-1H-pyrazolo[3,4-b]pyridin-4-amine (Compound No. 1),

N-cyclohexyl-1-ethyl-5-[(cis*)-8-(fluoromethyl)-1-oxa-2-azaspiro[4.5]dec-2-en-3-yl]-6-methyl-1H-pyrazolo[3,4-b]pyridin-4-amine (Compound No. 2),

(Cis*)-7-[4-(cyclohexylamino)-1-ethyl-6-methyl-1H-pyrazolo[3,4-b]pyridin-5-yl]-5-oxa-6-azaspiro[3.4]oct-6-ene-2-carbonitrile (Compound No. 3),

{(Cis*)-3-[4-(cyclohexylamino)-1-ethyl-6-methyl-1H-pyrazolo [3,4-b]pyridin-5-yl]-1-oxa-2-azaspiro[4.5]dec-2-en-8-yl}methanol (Compound No. 4),

(Trans*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylic acid (Compound No. 5),

(Cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylic acid (Compound No. 6),

N-cyclohexyl-1-ethyl-6-methyl-5-(1-oxa-2-azaspiro[4.4]non-2-en-3-yl)-1H-pyrazolo[3,4-b]pyridin-4-amine (Compound No. 7),

1-Ethyl-6-methyl-5-(1-oxa-2-azaspiro[4.4]non-2-en-3-yl)-N-(tetrahydro-2H-pyran-4-yl)-1H-pyrazolo[3,4-b]pyridin-4-amine (Compound No. 8),

1-Ethyl-6-methyl-5-(1-oxa-2-azaspiro[4.5]dec-2-en-3-yl)-N-(tetrahydro-2H-pyran-4-yl)-1H-pyrazolo[3,4-b]pyridin-4-amine (Compound No. 9),

N-cyclohexyl-1-ethyl-6-methyl-5-(1-oxa-2-azaspiro[4.5]dec-2-en-3-yl)-1H-pyrazolo[3,4-b]pyridin-4-amine (Compound No. 10),

1-Ethyl-6-methyl-5-(5-oxa-6-azaspiro[3.4]oct-6-en-7-yl)-N-(tetrahydro-2H-pyran-4-yl)-1H-pyrazolo[3,4-b]pyridin-4-amine (Compound No. 11),

Ethyl (cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylate (Compound No. 12),

Ethyl (trans*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylate (Compound No. 13),

{3-[4-(Cyclohexylamino)-1-ethyl-6-methyl-1H-pyrazolo [3,4-b]pyridin-5-yl]-4,5-dihydroisoxazole-5,5-diyl}dimethanol (Compound No. 14),

N-Cyclohexyl-1-ethyl-6-methyl-5-(5-oxa-6-azaspiro[3.4]oct-6-en-7-yl)-1H-pyrazolo[3,4-b]pyridin-4-amine (Compound No. 15),

5-{(Cis*)-2-[(benzyloxy)methyl]-5-oxa-6-azaspiro [3.4]oct-6-en-7-yl }-N-cyclohexyl-1-ethyl-6-methyl-1H-pyrazolo[3,4-b]pyridin-4-amine (Compound No. 16),

5-{(Trans*)-2-[(benzyloxy)methyl]-5-oxa-6-azaspiro[3.4]oct-6-en-7-yl}-N-cyclohexyl-1-ethyl-6-methyl-1H-pyrazolo[3,4-b]pyridin-4-amine (Compound No. 17),

{(Trans *)-3-[4-(cyclohexylamino)-1-ethyl-6-methyl-1H-pyrazolo [3 ,4-b]pyridin-5-yl]-1-oxa-2-azaspiro [4.5]dec-2-en-8-yl}methanol (Compound No. 18),

N-cyclohexyl-1-ethyl-5-[(cis*)-8-(methoxymethyl)-1-oxa-2-azaspiro[4.5]dec-2-en-3-yl]-6-methyl-1H-pyrazolo[3,4-b]pyridin-4-amine (Compound No. 19),

N-cyclohexyl-5-[(cis*)-8-(ethoxymethyl)-1-oxa-2-azaspiro[4.5]dec-2-en-3-yl]-1-ethyl-6-methyl-1H-pyrazolo[3,4-b]pyridin-4-amine (Compound No. 20),

Ethyl (trans*)-3-[1-ethyl-6-methyl-4-(tetrahydro-2H-pyran-4-ylamino)-1 H-pyrazolo[3,4-b]pyridin-5 -yl]-1-oxa-2-azaspiro[4.5]dec-2-ene-8-carboxylate (Compound No. 21),

Ethyl (cis*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylate (Compound No. 22),

5-{2-[(B enzyloxy)methyl]-5-oxa-6-azaspiro[3.4]oct-6-en-7-yl}-1-ethyl-6-methyl-N-(tetrahydro-2H-pyran-4-yl)-1H-pyrazolo[3,4-b]pyridin-4-amine (Compound No. 23),

(Trans*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 24),

(Cis*)- 3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 25),

N-cyclohexyl-1-ethyl-6-methyl-5-(1,9,12-trioxa-2-azadispiro[4.2.4.2]tetradec-2-en-3-yl)-1H-pyrazolo[3,4-b]pyridin-4-amine (Compound No. 26),

3-[4-(Cyclohexylamino)-1-ethyl-6-methyl-1H-pyrazolo [3,4-b]pyridin-5-yl]-1-oxa-2-azaspiro[4.5]dec-2-en-8-one (Compound No. 27),

N-cyclohexyl-1-ethyl-5-(1-oxa-2-azaspiro[4.5]dec-2-en-3-yl)-6-(trifluoromethyl)-1H-pyrazolo[3,4-b]pyridin-4-amine (Compound No. 28),

N-cyclohexyl-1-ethyl-5-(1-oxa-2-azaspiro[4.4]non-2-en-3-yl)-6-(trifluoromethyl)-1H-pyrazolo[3,4-b]pyridin-4-amine (Compound No. 29),

N-cyclohexyl-1-ethyl-5-(5-oxa-6-azaspiro[3.4]oct-6-en-7-yl)-6-(trifluoromethyl)-1H-pyrazolo[3,4-b]pyridin-4-amine (Compound No. 30),

{3-[4-(Cyclohexylamino)-1-ethyl-6-(trifluoromethyl)-1H-pyrazolo[3,4-b]pyridin-5-yl]-4,5-dihydro-1,2-oxazole-5,5-diyl}dimethanol (Compound No. 31),

(Trans*)-3-[4-(cyclohexylamino)-1-ethyl-6-(trifluoromethyl)-1H-pyrazolo[3,4-b]pyridin-5- yl]-1-oxa-2-azaspiro[4.5]dec-2-ene-8-carboxylic acid (Compound No. 32),

(Cis*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylic acid (Compound No. 33),

1-Ethyl-5-(5-oxa-6-azaspiro[3.4]oct-6-en-7-yl)-N-(tetrahydro-2H-pyran-4-yl)-6-(trifluoromethyl)-1H-pyrazolo[3,4-b]pyridin-4-amine (Compound No. 34),

1-Ethyl-5-(1-oxa-2-azaspiro[4.4]non-2-en-3-yl)-N-(tetrahydro-2H-pyran-4-yl)-6-(trifluoromethyl)-1H-pyrazolo[3,4-b]pyridin-4-amine (Compound No. 35),

1-Ethyl-5-(1-oxa-2-azaspiro [4.5]dec-2-en-3-yl)-N-(tetrahydro-2H-pyran-4-yl)-6-(trifluoromethyl)-1H-pyrazolo[3,4-b]pyridin-4-amine (Compound No. 36),

1-Ethyl-N-(tetrahydro-2H-pyran-4-yl)-6-(trifluoromethyl)-5-(1,9,12-trioxa-2-azadispiro[4.2.4.2]tetradec-2-en-3-yl)-1H-pyrazolo[3,4-b]pyridin-4-amine (Compound No. 37),

Ethyl (trans*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylate (Compound No. 38),

Ethyl (cis*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylate (Compound No. 39),

tert-butyl 3-[1-ethyl-4-(tetrahydro-2H-pyran-4-ylamino)-6-(trifluoromethyl)-1H-pyrazolo[3 ,4-b]pyridin-5-yl]-1-oxa-2,8-diazaspiro [4.5]dec-2-ene-8-carboxylate (Compound No. 40),

or their pharmaceutically acceptable salts, pharmaceutically acceptable solvates, stereoisomers, tautomers, racemates, regioisomers, geometric isomers, prodrugs, metabolites, polymorphs or N-oxides.

wherein * represent tentative regiochemical assignments.

The term “pharmaceutically acceptable” means approved by regulatory agency of the federal or a state government or listed in the U.S. Pharmacopoeia or other generally recognized pharmacopoeia for use in mammals, and more particularly in humans.

The term “pharmaceutically acceptable salts” refers to the derivates of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The term “pharmaceutically acceptable solvates” refers to solvates with water such as hydrates, hemihydrate or sesquihydrate, or pharmaceutically acceptable solvents, for example solvates with common organic solvents as ethanol and the like. Such solvates are also encompassed within the scope of the disclosure.

The present invention also includes within its scope prodrugs of these agents. In general, such prodrugs will be functional derivatives of these compounds, which are readily convertible in vivo into the required compound. Conventional procedures for the selection and preparation of prodrugs are known.

The disclosed compounds may get metabolized in vivo and these metabolites are also encompassed within the scope of this invention.

The term “polymorphs” includes all crystalline form as well as amorphous form for compounds described herein and are included in the present invention.

All stereoisomers of the compounds of the invention are contemplated, either in admixture or in pure form. The compounds of the present invention can have asymmetric centers at any of the carbon atoms including all the substituents. Consequently, compounds of present invention can exist in enantiomeric or diastereomeric forms or in mixture thereof. The processes for the preparation can utilize racemates, enantiomers, or diastereomers as starting materials. When diastereomeric or enantiomeric products are prepared, they can be separated by conventional methods, for example, chromatographic or fractional crystallization.

The term “tautomer” includes one of two or more structural isomers that exist in equilibrium and are readily converted from one isomeric form to another. Certain compounds of the invention may furthermore be present in tautomeric forms.

The term “regioisomers” refers to compounds, which have the same molecular formula but differ in the connectivity of the atoms.

The term, “geometric isomers”, refers to compounds, having the same molecular formula as another but a different geometric configuration, as when atoms or groups of atoms are attached in different spatial arrangements on either side of a double bond or other rigid bond.

The term “racemate” includes a mixture of equal amounts of left- and right-handed stereoisomers of chiral molecules.

In another aspect, the present invention includes pharmaceutical compositions comprising, as an active ingredient, at least one of the disclosed compound or a pharmaceutically acceptable salt, a pharmaceutically acceptable solvate, stereoisomer, tautomer, racemate, regioisomer, geometric isomer, prodrug, metabolite, polymorph or N-oxide, along with a pharmaceutically acceptable carrier, excipient or diluent. Compounds disclosed herein may be administered to mammal for treatment by any route, which effectively transports the active compound to the appropriate or desired site of action such as oral, nasal, pulmonary, transdermal or parenteral (rectal, subcutaneous, intravenous, intraurethral, intramuscular, intranasal). The pharmaceutical composition of the present invention comprises a pharmaceutically effective amount of a compound of the present invention formulated along with one or more pharmaceutically acceptable carriers, excipients or diluents. The choice of pharmaceutical carrier, excipient or diluent can be made with regard to the intended route of administration and standard pharmaceutical practice.

Where desired, the compounds of the invention and/or their pharmaceutically acceptable salts, pharmaceutically acceptable solvates, stereoisomers, tautomers, racemates, regioisomers, geometric isomers, prodrugs, metabolites, polymorphs or N-oxides may be advantageously used in combination with one or more other compounds.

Examples of other compounds, which may be used in combination with compounds of this invention and/ or their pharmaceutically acceptable salts, pharmaceutically acceptable solvates, stereoisomers, tautomers, racemates, regioisomers, geometric isomers, prodrugs, metabolites, polymorphs or N-oxides include β2- agonists, corticosteroids, leukotriene antagonists, 5-lipoxygenase inhibitors, chemokine inhibitors, p38 kinase inhibitors, anticholinergics, antiallergics, PAF (platelet activating factor) antagonists, EGFR (epidermal growth factor receptor) kinase inhibitors, muscarinic receptor antagonists or combination(s) thereof.

The one or more β2-agonist as described herein may be chosen from those described in the art. The β2-agonists may include one or more compounds described in U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258.

β2-agonists include, for example, one or more of albuterol, salbutamol, biltolterol, pirbuterol, levosalbutamol, tulobuterol, terbutaline, bambuterol, metaproterenol, fenoterol, salmeterol, carmoterol, arformoterol, formoterol, and their pharmaceutically acceptable salts or solvates thereof.

Corticosteroids as described herein may be chosen from those described in the art. Corticosteroids may include one or more compounds described in U.S. Pat. Nos. 3,312,590; 3,983,233; 3,929,768; 3,721,687; 3,436,389; 3,506,694; 3,639,434; 3,992,534; 3,928,326; 3,980,778; 3,780,177; 3,652,554; 3,947,478; 4,076,708; 4,124,707; 4,158,055; 4,298,604; 4,335,121; 4,081,541; 4,226,862; 4,290,962; 4,587,236; 4,472,392; 4,472,393; 4,242,334; 4,014,909; 4,098,803; 4,619,921; 5,482,934; 5,837,699; 5,889,015; 5,278,156; 5,015,746; 5,976,573; 6,337,324; 6,057,307; 6,723,713; 6,127,353; and 6,180,781.

Corticosteroids may include, for example, one or more of alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, methylprednisolone, mometasone, prednicarbate, prednisolone, rimexolone, tixocortol, triamcinolone, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone. Examples of possible salts or derivatives include: sodium salts, sulfobenzoates, phosphates, isonicotinates, acetates, propionates, dihydrogen phosphates, palmitates, pivalates, or furoates. In some cases, the corticosteroids may also occur in the form of their hydrates.

The leukotriene antagonist can be selected from compounds, for example, those described in U.S. Pat. Nos. 5,565,473; 5,583,152; 4,859,692 or 4,780,469.

Examples of leukotriene antagonist include, but are not limited to, montelukast, zafirlukast, pranlukast and pharmaceutically acceptable salts thereof.

5-Lipoxygenase inhibitors can be selected from, for example, compounds in U.S. Pat. Nos. 4,826,868, or 4,873,259, or European Patent Nos. 0 419 049, 0 542 356 or 0 542 355. Examples may include, but are not limited to, atreleuton, zyflo (zileuton), ABT-761, fenleuton or tepoxalin.

Examples of the chemokine inhibitors include, but are not limited to, endogenous ligands of chemokine receptors or derivatives thereof, and non-peptidic low molecular compounds or antibodies for chemokine receptors.

Examples of the endogenous ligands of chemokine receptors include, but are not limited to, MIP-1α, MIP-1β, Rantes, SDF-1α, SDF-1β, MCP-1, MCP-2, MCP4, Eotaxin, and MDC. Examples of the derivatives of endogenous ligands include, but are not limited to, AOP-RANTES, Met-SDF-1α, and Met-SDF-1β.

Examples of the antibodies for chemokine receptors include, but are not limited to, Pro-140.

Examples of the non-peptidic low molecular compounds include, but are not limited to, antagonists and agonists for CCR1, CCR2, CCR3, CCR4, CCR5, CXCR1, CXCR2, CXCR3 and CXCR4 receptors.

p38 kinase inhibitors include compounds disclosed in WO 2006/021848, WO 2006/016237, WO 2006/056863, WO 2006/117657 and WO 2006/082492. Any reference to the above mentioned p38 kinase inhibitors also include any pharmacologically acceptable salts thereof which may exist.

Anticholinergics include, for example, tiotropium salts, ipratropium salts, oxitropium salts, salts of the compounds known from WO 02/32899: tropenol N-methyl-2,2-diphenylpropionate, scopine N-methyl-2,2-diphenylpropionate, scopine N-methyl-2-fluoro-2,2-diphenylacetate and tropenol N-methyl-2-fluoro-2,2-diphenylacetate; as well as salts of the compounds known from WO 02/32898: tropenol N-methyl-3,3′,4,4′-tetrafluorobenzilate, scopine N-methyl-3,3′,4,4′-tetrafluorobenzilate, scopine N-methyl-4,4′-dichlorobenzilate, scopine N-methyl-4,4′-difluorobenzilate, tropenol N-methyl-3,3′-difluorobenzilate, scopine N-methyl-3,3′-difluorobenzilate, and tropenol N-ethyl-4,4′-difluorobenzilate, optionally in the form of their hydrates and solvates. By salts are meant those compounds which contain, in addition to the above mentioned cations, as counter-ion, an anion with a single negative charge selected from among the chloride, bromide, and methanesulfonate.

Preferred anticholinergics include, for example, tiotropium bromide, ipratropium bromide, oxitropium bromide, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide, scopine 4,4′-dichlorobenzilate methobromide, scopine 4,4′-difluorobenzilate methobromide, tropenol 3,3′-difluorobenzilate methobromide, scopine 3,3′-difluorobenzilate methobromide, and tropenol 4,4′-difluorobenzilate ethylbromide.

Antiallergics include, for example, epinastine, cetirizine, azelastine, fexofenadine, levocabastine, loratadine, mizolastine, ketotifene, emedastine, dimetindene, clemastine, bamipine, hexachloropheniramine, pheniramine, doxylamine, chlorophenoxamine, dimenhydrinate, diphenhydramine, promethazine, ebastine, desloratadine, and meclizine. Preferred antiallergic agents include, for example, epinastine, cetirizine, azelastine, fexofenadine, levocabastine, loratadine, ebastine, desloratadine, and mizolastine. Any reference to the above-mentioned antiallergic agents also includes any pharmacologically acceptable salts thereof, which may exist.

PAF antagonists include, for example, 4-(2-chlorophenyl)-9-methyl-2-[3-(4-morpholinyl)-3-propanon-1-yl]-6H-thieno [3,2-f][1,2,4]triazolo [4,3-α][1,4]diazepine and 6-(2-chlorophenyl)-8,9-dihydro-1-methyl-8-[(4-morpholinyl)carbonyl]-4H,7H-cyclopenta[4.5]thieno[3,2-f][1,2,4]triazolo[4,3-α][1,4]diazepine.

EGFR kinase inhibitors include, for example, 4-[(3-chloro-4-fluorophenyl)amino]-7-(2-{4-[(S)-(2-oxotetrahydrofuran-5-yl)carbonyl]piperazin-1-yl}-ethoxy)-6-[(vinylcarbonyl)amino]quinazoline, 4-[(3-chloro4-fluorophenyl)amino]-7-[4-((S)-6-methyl-2- oxomorpholin-4-yl)butyloxy]-6-[(vinylcarbonyl)amino]quinazoline, 4-[(3-chloro4-fluorophenyl) amino]-7-[44(R)-6-methyl-2-oxomorpholin-4-yObutyloxy]-6-[(vinylcarbonyl)amino]quinazoline, 4-[(3-chloro-4-fluorophenyl)amino]-7-[24(S)-6-methyl-2-oxomorpholin-4-yl)ethoxy]-6-[(vinylcarbonypamino]quinazoline, 4-[(3-chloro-4-fluorophenyl) amino]-6- [(4-{N-[2-(ethoxycarbonypethyl]-N- [(ethoxycarbonyl)methyl]-amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-l-yl]amino}-7-cyclopropyl-methoxyquinazoline, and 4-[(3-chloro-4-fluorophenypamino]-6-[3-(morpholin-4-yl)propyloxy]-7-methoxyquinazoline. Any reference to the above-mentioned EGFR kinase inhibitors also includes any pharmacologically acceptable salts thereof which may exist.

Muscarinic receptor antagonists include substances that directly or indirectly block activation of muscarinic cholinergic receptors. Examples include, but are not limited to, quaternary amines (e.g., methantheline, ipratropium, propantheline), tertiary amines (e.g., dicyclomine, scopolamine) and tricyclic amines (e.g., telenzepine). Other muscarinic receptor antagonists include benztropine, hexahydro-sila-difenidol hydrochloride (HHSID hydrochloride), (+/−)-3-quinuclidinyl xanthene-9-carboxylate hemioxalate (QNX-hemioxalate), telenzepine dihydrochloride, tolterodine, oxybutynin and atropine.

Examples set forth below demonstrate the synthetic procedures for the preparation of the representative compounds. The examples are provided to illustrate particular aspect of the disclosure and do not constrain the scope of the present invention.

### EXPERIMENTAL DETAILS

### Example 1

### Preparation of tetrahydro-2H-pyran-4-amine hydrochloride

This compound was synthesized according to the procedure reported in Tetrahedron letters, 42, 4257-4259, (2001).

Yield: 79.20%

m/z: (M⁺+1) 101.81

### Example 2

### Preparation of 1-ethyl-1H-pyrazol-5-amine

Hydrazine hydrate (98.7 gm, 1.97 moles) was added drop wise to a solution of acrylonitrile (100 gm, 1.88 moles) in tetrahydrofuran (350 ml) at 0-5° C. The reaction mixture was stirred at room temperature for about 3 hrs and acetaldehyde (86.9 gm, 1.9 moles) was added drop wise followed by stirring at room temperature for about 3 hrs. The solvent was removed at reduced pressure to give yellow oil, which was dissolved in n-butanol (100 ml) and further, n-butanol (250 ml) containing potassium t-butoxide (221 gm, 1.9 moles) was added. The resulting mixture was gradually heated from room temperature to 120° C. and heating was continued for about 3 hrs. The reaction mixture was cooled to room temperature and water (500 ml) was added. Extraction was done with ethyl acetate (3×500 ml). The organic layer was dried over anhydrous sodium sulfate and concentrated in vacuo. The crude compound was purified by column chromatography using silica gel (100-200 mesh) saturated with triethyl amine. Elution was done by using 20% ethyl acetate in hexane.

Yield: 66.0 gm (15%)

m/z: (M⁺+1) 111.98

### Example 3

### Preparation of 2-(1-ethoxy-ethylidene)-malonic acid diethyl ester

This compound was prepared according to the procedure reported in Journal of Medicinal Chemistry, 49, 21, 6351-6363, (2006).

Yield: 59.6%

m/z: (M⁺+1) 231.14

### Example 4

### Preparation of ethyl 4-chloro--ethyl-6-methyl-1H-pyrazolo13,4-bl pyridine-5-carboxylate

A mixture of 1-ethyl-1H-pyrazol-5-amine (16 gm, 0.144 moles) (example 2) and 2-(1-ethoxy-ethylidene)-malonic acid diethyl ester (29 gm, 0.12 moles) (example 3) was stirred at 120° C. for about 2 hrs. When the reaction was complete on TLC, crude reaction mixture was concentrated in vacuo to remove ethanol, which was formed as byproduct. Crude residue was taken in phosphorous oxychloride (50 ml) and heated at 115° C. for about 12-13 hrs. The reaction mixture was poured into ice-cooled water with continuous stirring. Solid which precipitated out was filtered and dried under vacuum to afford title compound as creamy white solid.

Yield: 12 gm (57.17%)

m/z: (M⁺+1) 268.28

### Example 4 (a)

### Preparation 1-2,2,2-trifluoroacetamide

A solution of trifluoro acetic anhydride (10.40 gm, 0.0495 moles) was added dropwise to a solution of 1-ethyl-1H-pyrazol-5-amine (6.0 gm, 0.045 moles) (example 2) in dichloromethane (50 ml) at 0° C. After completion of addition, ice bath was removed and reaction mixture was stirred for about 3 hrs at room temperature. Saturated sodium bicarbonate solution was added and the mixture was stirred for about 10 minutes. Organic layer was separated and washed with water and brine, dried and concentrated in vacuo to give crude product, which was purified by column chromatography over silica gel. Elution was done by using 25% ethyl acetate in hexane.

Yield: 6.1 gm (80%)

m/z: (M⁺+1) 208.5

### Example 4 (b)

### Preparation of (1 Z)-N-(1-ethyl-1H-pyrazol-5-yl)-2,2,2-trifluoroethanimidoyl chloride

Triphenylphosphine (1.39 gm, 0.00531 moles) was added to a solution of N-(1-ethyl-1H-pyrazol-5-yl)-2,2,2-trifluoroacetamide (1.0 gm, 0.00483 moles) (example 4 (a)) in carbon tetrachloride (20 ml). The reaction mixture was refluxed for about 12 hrs. It was cooled at room temperature, diluted with dichloromethane and washed with water and brine. The organic layer was dried over anhydrous sodium sulphate and concentrated under vacuo to get crude mixture, which was used as such for further step.

### Example 4 (c)

### Preparation of diethyl {1-[1(1-ethyl-1H-nyrazol-5-yl)amino]-2,2,2-trifluoroethylidene}propanedioate

Sodium hydride (250 mg, 0.00724 moles) was added to a solution of diethyl malonate (850 mg, 0.00531 moles) in tetrahydrofuran (10 ml) at 0° C. The reaction mixture was stirred at room temperature for about 30 minutes. It was cooled again to 0° C. and solution of (1Z)-N-(1-ethyl-1H-pyrazol-5-yl)-2,2,2-trifluoroethanimidoyl chloride (2.5 gm crude) (example 4 (b)) in tetrahydrofuran (25 ml) was added gradually. After completion of addition, the reaction mixture was stirred at room temperature for about 18 hrs. The reaction was quenched by slow addition of water at 0° C. The solvent was removed and ethyl acetate was added. Extraction was done with water. The organic layer was dried over anhydrous sodium sulphate and concentrated under vacuo to get crude mixture which was purified by column chromatography. Elution was done by using 5% ethyl acetate in hexane.

Yield: 400 mg

m/z: (M⁺+1) 350.93

### Example 4 (d)

### Preparation of ethyl 1-ethyl-4-hydroxy-6-(trifluoromethyl)-1H-pyrazolo[3,4-b]pyridine-5-carboxylate

Diethyl {1- [(1-ethyl-1H-pyrazol-5-yl(amino]-2,2,2-trifluoroethylidene}propanedioate (4.5 gm, 0.0129 moles) (example 4 (c)) was dissolved in toluene (50 ml) and the mixture was refluxed for about 15 hrs. The solvent was removed under reduced pressure and crude product was purified by column chromatography over silica gel. Elution was done by using 50% ethyl acetate in hexane.

Yield: 2.2 gm (56%)

m/z: (M⁺+1) 304.58

Example 4 (e): Preparation of ethyl 4-chloro-1-ethyl-6-(trifluoromethyl)-1H-pyrazolo[3,4-b]pyridine-5-carboxylate

Phosphorus oxy chloride (500 mg, 0.0033 moles) was added to ethyl 1-ethyl-4-hydroxy-6-(trifluoromethyl)-1H-pyrazolo[3,4-b]pyridine-5-carboxylate (500 mg, 0.0016 moles) (example 4(d)) and the mixture was heated at 110° C. for about 24 hrs. It was diluted with dichloromethane and poured over crushed ice. The organic layer was separated, concentrated and purified over silica gel (elution was done using 10% ethyl acetate in hexane) to give the title compound.

Yield: 28%

m/z: (M⁺1) 322.65

### Example 4 (f)

### Preparation of ethyl 4-chloro-1-ethyl-6-(trifluoromethyl)-1H-pyrazolo[3,4-b]pyridine-5-carboxylate

The title compound was also prepared by reacting diethyl {1-[(1-ethyl-1H-pyrazol-5-yl)amino]-2,2,2-trifluoroethylidene}propanedioate (example 4 (c)) (3.0 gm, 0.0085 moles) with phosphorus oxy chloride (1.60 ml, 0.0172 moles) by following the procedure given in example 4 (e).

Yield: 52%

### Examle 5

### Preparation of ethyl 4-(cyclohexylamino)-1-ethyl-6-methyl-1H-pyrazolo [3,4-b]pyridine-5-carboxylate

Cyclohexyl amine (30 gm, 0.1137 moles) was added to a mixture of ethyl 4-chloro-1-ethyl-6-methyl-1H-pyrazolo [3,4-b]pyridine-5-carboxylate (38 ml, 0.331 moles) (example 4) in acetonitrile (100 ml). After stirring for about 2 h at 110° C., acetonitrile was removed under reduced pressure. Water was added and the reaction mixture was extracted with ethyl acetate. The organic layer was washed with brine, dried over anhydrous sodium sulfate and concentrated in vacuo to give brownish solid.

Yield: 35 gm (94.41%),

m/z: (M⁺+1) 331.39

The following compounds were prepared similarly

Ethyl 1-ethyl-6-methyl-4-(tetrahydro-2H-pyran-4-ylamino)-1H-pyrazolo[3,4-b]pyridine-5-carboxylate

m/z: (M⁺+1) 333.73

Ethyl 4-(cyclohexylamino)-1-ethyl-6-(trifluoromethyl)-1H-pyrazolo[3,4-b]pyridine-5-carboxylate

m/z: (M⁺+1) 394.98

Ethyl 1-ethyl-4-(tetrahydro-2H-pyran-4-ylamino)-6-(trifluoromethyl)-1H-pyrazolo[3,4-b]pyridine-5-carboxylate

m/z: (M⁺+1)386.80

### Example 6

### Preparation of 4-cyclohexylamino-1-ethyl-6-methyl-1H-pyrazolo [3,4-b]pyridine-5-carboxylic acid

Sodium hydroxide solution (16.9 gm, 0.106 moles in 100 ml water) was added to a solution of ethyl 4-(cyclohexylamino)-1-ethyl-6-methyl-1H-pyrazolo[3,4-b]pyridine-5-carboxylate (35 gm, 0.106 moles) (example 5) in ethanol (150 ml). The reaction mixture was stirred for about 14 hrs at room temperature. Ethanol was removed under reduced pressure. Water was added and the reaction mixture was extracted with ethyl acetate. Aqueous layer was acidified by using hydrochloric acid (2N) to pH of about 4-5. White solid, which was obtained, was filtered and dried under vacuo.

Yield: 30 gm (crude) (93.66%)

m/z: (M⁺+1) 303.44

The following compounds were prepared similarly

1-Ethyl-4-(tetrahydro-2H-pyran-4-ylamino)-6-methyl-1H-pyrazolo[3,4-b]pyridine-5-carboxylic acid

m/z: (M⁺+1) 305.65

1-Ethyl-4-(tetrahydro-2H-pyran-4-ylamino)-6-(trifluoromethyl)-1H-pyrazolo[3,4-b]pyridine-5-carboxylic acid

m/z: (M⁺+1) 358.78

4-(Cyclohexylamino)-1-ethyl-6-(trifluoromethyl)-1H-pyrazolo[3,4-b]pyridine-5-carboxylic acid

m/z: (M⁺+1) 356.90

### Example 7

### Preparation of 4-(cyclohexylamino)-1-ethyl-6-methyl-N-methoxy-N-methyl-1H-pyrazolo[3,4-b]pyridine-5-carboxamide

4-Cyclohexylamino-1-ethyl-6-methyl-1H-pyrazolo[3,4-b]pyridine-5-earboxylic acid (30.0 gm, 0.099 moles) (example 6) and N,O-dimethylhydroxylamine hydrochloride (14 gm, 0.149 moles) were taken in dimethylformamide (200 ml). At 0° C., hydroxybenzotriazole (26 gm, 0.198 moles) and N-methylmorpholine (65 ml, 0.594 moles) were added and the reaction mixture was stirred for about 1 hr. 1-Ethyl-3-(3-dimethylaminopropyl) carbodiimide hydrochloride (37 gm, 0.198 moles) was added and the reaction mixture was stirred for about 14 hrs. Water was added and extraction was carried out with ethyl acetate. The organic layer was washed with brine, dried over anhydrous sodium sulfate and concentrated in vacuo. The compound was purified over column chromatography using 40% ethyl acetate in hexane.

Yield: 28 gm (81.70%)

m/z: (M⁺+1) 346.76

The following compounds were prepared similarly

1-Ethyl-N-methoxy-N-methyl-4-(tetrahydro-2H-pyran-4-ylamino)-6-methyl-1 H-pyrazolo[3,4-b]pyridine-5-carboxamide

m/z: (M⁺+1) 348.14

4-(Cyclohexylamino)-1-ethyl-N-methoxy-N-methyl-6-(trifluoromethyl)-1H-pyrazolo[3,4-b]pyridine-5-carboxamide

m/z: (M⁺+1) 400.03

1-Ethyl-N-methoxy-N-methyl-4-(tetrahydro-2H-pyran-4-ylamino)-6-(trifluoromethyl)-1H-pyrazolo[3,4-b]pyridine-5-carboxamide

### Example 8

### Preparation of 4-(cyclohexylamino)-1-ethyl-6-methyl-1H-pyrazolo[3,4-b]pyridine-5-carbaldehyde

Toluene (50 ml) was cooled to −30 to −35° C. and vitride (31.6 ml, 0.162 moles) was added. After about 10 min , 4-(cyclohexylamino)-1-ethyl-6-methyl-N-methoxy-N-methyl-1H-pyrazolo[3,4-b]pyridine-5-carboxamide (28 gm, 0.081moles) (example 7) was added and the reaction mixture was stirred for about 4 hrs. Citric acid (10%) solution was added dropwise to quench the reaction and the reaction mixture was extracted with ethyl acetate. The organic layer was washed with brine and dried over anhydrous sodium sulfate and concentrated in vacuo. The compound was purified over column chromatography using 60% ethyl acetate in hexane.

Yield: 20 gm (86.16%).

m/z: (M⁺+1) 287.72

The following compounds were prepared similarly

1-Ethyl-4-(tetrahydro-2H-pyran-4-ylamino)-6-methyl-1H-pyrazolo[3,4-b]pyridine-5-carbaldehyde

m/z: (M⁺+1) 289.66

4-(Cyclohexylamino)-1-ethyl-6-(tifluoromethyl)-1H-pyrazolo[3,4-b]pyridine-5-carbaldehyde

m/z: (M⁺+1) 342.86

1-Ethyl-4-(tetrahydro-2H-pyran-4-ylamino)-6-(trifluoromethyl)-1H-pyrazolo[3,4-b]pyridine-5-carbaldehyde

m/z: (M⁺+1) 344.8

### Example 9

### Preparation of 4-(cyclohexylamino)-1-ethyl-6-methyl-1H-pyrazolo[3,4-b]pyridine-5-carbaldehyde oxime

Hydroxylamine hydrochloride (7.2 gm, 0.117 moles) and sodium acetate (8.4 gm, 0.117 moles) were added to a stirred solution of 4-(cyclohexylamino)-1-ethyl-6-methyl-1H-pyrazolo[3,4-b]pyridine-5-carbaldehyde (6 gm, 0.0234 moles) (example 8) in ethanol (150 ml). The reaction mixture was allowed to stir at room temperature for about 14 hrs. Ethanol was removed under reduced pressure and residue was poured in water. The reaction mixture was extracted with ethyl acetate. The organic layer was washed with brine and dried over anhydrous sodium sulfate and concentrated in vacuo.

Yield: 5.5gm (87.16%)

m/z: (M⁺+1) 302.69

The following compounds were prepared similarly

1-Ethyl-4-(tetrahydro-2H-pyran-4-ylamino)-6-methyl-1H-pyrazolo[3,4-b]pyridine-5-carbaldehyde oxime

m/z: (M⁺+1) 304.27

1-Ethyl-5-[(E)-(hydroxyimino)methyl]-N-(tetrahydro-2H-pyran-4-yl)-6-(trifluoromethyl)-1H-pyrazolo[3,4-b]pyridin-4-amine

N-cyclohexyl-1-ethyl-5-[(E)-(hydroxyimino)methyl]-N-(trifluoromethyl)-1H-pyrazolo[3,4-b]pyridin-4-amine

### Example 10

### Preparation of N-cyclohex 1-o) iro 4.4 non-2-en-3-yl)-1H-pyrazolo[3,4-b]pyridin-4-amine (Compound No. 7)

Methylene cyclopentane (0.048 ml, 0.00046 moles) was added to 4-(cyclohexylamino)-1-ethyl-6-methyl-1H-pyrazolo[3,4-b]pyridine-5-carbaldehyde oxime (70 mg, 0.00023 moles) (example 9) in mixture of dichloromethane : chloroform (1:1, 10 ml). The reaction mixture was stirred at room temperature for about 5 minutes. Sodium hypochlorite (0.7 ml) was added slowly to the reaction mixture over a period of about 5 minutes and the mixture was allowed to stir at room temperature for about 14 hrs. The organic solvent was evaporated and the residue was extracted in chloroform. The organic layer was concentrated and the title compound obtained was purified by preparative thin layer chromatography using 60% ethyl acetate in hexane.

Yield: 20 mg (22.73%)

m/z: (M⁺+1) 382.30

¹HNMR (δ, CDCl₃): 7.89 (1H, s), 6.75-6.77 (1H, d), 4.41-4.47 (2H, q), 3.83-3.85 (1H, t), 3.21 (2H, s), 2.52 (3H, s), 2.10-2.17 (4H, m), 1.87-1.9 (2H, m), 1.74-1.77 (3H, t), 1.41-1.72 (12H, m)

The following compounds were prepared similarly

1-Ethyl-6-methyl-5-(1-oxa-2-azaspiro[4.4]non-2-en-3-yl)-N-(tetrahydro-2H-pyran-4-yl)-1H-pyrazolo[3,4-b]pyridin-4-amine (Compound No. 8),

m/z: (M⁺+1) 384.18

¹HNMR (δ, CDCl₃): 7.89 (1H, s), 7.00 (1H, bs), 4.42-4.48 (2H, q), 4.02 (1H, s), 3.99 (2H, bs), 3.57-3.6 (2H, m), 3.23 (2H, s), 2.54 (3H, s), 2.10-2.13 (6H, m), 1.68-1.78 (6H, m), 1.47-1.49 (3H, t)

1-Ethyl-6-methyl-5-(1-oxa-2-azaspiro[4.5]dec-2-en-3-yl)-N-(tetrahydro-2H-pyran-4-yl)-1H-pyrazolo[3,4-b]pyridin-4-amine (Compound No. 9),

m/z: (M⁺+1) 398.29; Purity: 86.37%

¹HNMR (δ, CDCl₃): 7.88 (1H, s), 6.92 (1H, bs), 4.42-4.47 (2H, q), 3.98-4.11 (3H, m), 3.57-3.64 (2H, m), 3.03 (2H, s), 2.53 (3H, s), 1.84-2.12 (14H, m), 1.49- 1.59 (3H, t)

N-cyclohexyl-1-ethyl-6-methyl-5-(1-oxa-2-azaspiro[4.5]dec-2-en-3-yl)-1H-pyrazolo[3,4 -b]pyridin- 4 -amine (Compound No. 10), m/z: (M⁺+1) 396.19

¹HNMR (δ, CDCl₃): 7.89 (1H, s), 6.67 (1H, bs), 4.41-4.47 (2H, q), 3.83 (1H, bs), 3.01 (2H, s), 2.51 (3H, s), 2.10-2.30 (2H, m), 1.78-1.84 (6H, m), 1.61-1.72 (6H, m), 1.25-1.50 (9H, m)

1-Ethyl-6-methyl-5-(5-oxa-6-azaspiro[3.4]oct-6-en-7-yl)-N-(tetrahydro-2H-pyran-4-yl)-1H-pyrazolo[3,4-b]pyridin-4-amine (Compound No. 11),

m/z: (M⁺+1) 370.30; Purity: 93.80%

¹HNMR (δ, CDCl₃): 7.88 (1H, s), 6.9 (1H, bs), 4.42-4.47 (2H, q), 3.98-4.0 (3H, m), 3.57-3.61 (311, t), 3.35 (2H, s), 2.54-2.58 (5H, m), 2.08-2.24 (4H, m), 1.55-1.85 (3H, m), 1.47-1.51 (3H, t) —{3-[4-(Cyclohexylamino)-1-ethyl-6-methyl-1H-pyrazolo[3,4-b]pyridin-5-yl]-4,5-dihydroisoxazole-5,5-diyl}dimethanol (Compound No. 14), m/z: (M⁺+1) 388.21

¹HNMR (δ, CDCl₃): 7.87 (1H, s), 5.69-5.7 (1H, d), 4.42-4.47 (2H, q), 3.86-3.89 (2H, d), 3.78 (1H, bs), 3.75-3.77 (2H, q), 3.27 (2H, s), 2.17 (3H, s), 2.09-2.12 (2H, d), 1.71-1.82 (2H, m), 1.61- 1.66 (3H, t), 1.18-1.51 (8H, m) - N-Cyclohexyl-1-ethyl-6-methyl-5-(5-oxa-6-azaspiro[3.4]oct-6-en-7-yl)-1H-pyrazolo[3,4-b]pyridin-4-amine (Compound No. 15), m/z: (M⁺+1) 368.19

¹HNMR (δ, CDCl₃): 7.89 (1H, s), 6.74 (1H, bs), 4.41-4.47 (2H, q), 3.83 (1H, bs), 3.33 (2H, s), 2.55-2.60 (5H, m), 2.21-2.26 (2H, m), 2.07-2.10 (2H, m) 1.84-1.92 (2H, m), 1.63-1.68 (5H, m), 1.27-1.51 (6H, m)

5-{2-[(Benzyloxy)methyl]-5-oxa-6-azaspiro[3.4]oct-6-en-7-yl}-1-ethyl-6-methyl-N-(tetrahydro-2H-pyran-4-yl)-1H-pyrazolo[3,4-b]pyridin-4-amine (Compound No. 23), m/z: (M⁺+1) 490.93

¹HNMR (δ, CDCl₃): 7.87 (1H, s), 7.28-7.35 (5H, m), 4.53 (2H, s), 4.42-4.47 (2H, m), 3.96-4.00 (3H, m), 3.49-3.57 (4H, m), 3.26 (2H, s), 2.66-2.68 (3H, m), 2.47 (3H, m), 2.17-2.19 (2H, m), 1.25-2.11 (8H, m)

N-cyclohexyl-1-ethyl-6-methyl-5-(1,9,12-trioxa-2-azadispiro[4.2.4.2]tetradec-2-en-3-yl)-1H-pyrazolo[3,4-b]pyridin-4-amine (Compound No. 26), m/z: (M⁺+1) 454.92

¹HNMR (δ, CDCl₃): 7.89 (1H, s), 6.75 s), 4.41-4.46 (2H, q), 3.98-4.01 (4H, m), 3.84-3.97 (1H, m), 3.06 (2H, s), 2.51 (3H, s), 1.25-2.08 (21H, m) - N-cyclohexyl-1-ethyl-5-(1-oxa-2-azaspiro[4.5]dec-2-en-3-yl)-6-(trifluoromethyl)-1H-pyrazolo[3,4-b]pyridin-4-amine (Compound No. 28), m/z: (M⁺+1) 450.02

¹HNMR (δ, CDCl₃): 7.95 (1H , s), 6.12-6.16 (1H, m), 4.47-4.51 (2H, m), 3.84-3.88 (1H, m), 2.91 (2H, s), 2.60-2.63 (2H, m), 2.10-2.22 (4H, m), 1.56-1.79 (12H, m), 1.34-1.37 (2H, m).

- N-cyclohexyl-1-ethyl-5-(1-oxa-2-azaspiro[4.4]non-2-en-3-yl)-6-(trifluoromethyl)-1H-pyrazolo[3,4-b]pyridin-4-amine (Compound No. 29),

m/z: (M⁺+1) 435.91

¹HNMR (δ, CDCl₃): 7.95 (1H, s), 6.20-6.23 (1H-, m), 4.46-4.51 (2H, m), 3.82-3.88 (1H, m), 3.11-3.13 (2H, m), 2.60-2.63 (2H, m), 2.15-2.22 (4H, m), 1.58-1.88 (10H, m), 1.46-1.51 (3H, m), 1.33-1.37 (2H, m).

N-cyclohexyl-1-ethyl-5-(5-oxa-6-azaspiro[3.4]oct-6-en-7-yl)-6-(trifluoromethyl)-1H-pyrazolo[3,4-b]pyridin-4-amine (Compound No. 30),

m/z: (M⁺+1) 422.00

¹HNMR (δ, CDCl₃): 7.95 (1H, s), 6.14-6.16 (1H, m), 4.47-4.53 (2H, m), 3.86-3.92 (1H, m), 3.22 (2H, s), 2.54-2.63 (2H, m), 2.08-2.34 (4H, m), 1.59-1.78 (8H, m), 1.49-1.53 (3H, m), 1.33-1.36 (2H, m).

{3-[4-(Cyclohexylamino)-1-ethyl-6-(trifluoromethyl)-1H-pyrazolo [3,4-b]pyridin-5-yl]-4,5-dihydro-1,2-oxazole-5,5-diyl}dimethanol (Compound No. 31),

m/z: (M⁺+1) 441.83

¹HNMR (δ, CDCl₃): 7.95 (1H, s), 5.84-5.86 (1H, d), 4.48-4.53 (2H, m), 3.88-3.92 (2H, m), 3.77-3.79 (1H, m), 3.64-3.69 (2H, m), 3.24 (2H, s), 2.04-2.18 (4H, m), 1.69-1.83 (4H, m), 1.49-1.52 (3H, m), 1.34-1.38 (2H, m).

1-Ethyl-5-(5-oxa-6-azaspiro[3.4]oct-6-en-7-yl)-N-(tetrahydro-2H-pyran-4-yl)-6-(trifluoromethyl)-1H-pyrazolo[3,4-b]pyridin-4-amine (Compound No. 34),

m/z: (M⁺+1) 424.43

¹HNMR (δ, CDCl₃): 7.98 (1H, s), 6.33-6.34 (1H, d), 4.49-4.54 (2H, m), 4.08-4.15 (2H, m), 3.99-4.02 (2H, m), 3.56-3.63 (2H, m), 3.26 (2H, s), 2.54-2.63 (2H, m), 2.22-2.28 (2H, m), 2.09-2.13 (2H, m), 1.86-1.91 (2H, m), 1.65-1.71 (2H, m), 1.49-1.53 (3H, t).

1-Ethyl-5-(1-oxa-2-azaspiro[4.4]non-2-en-3-yl)-N-(tetrahydro-2H-pyran-4-yl)-6-(trifluoromethyl)-1H-pyrazolo[3,4-b]pyridin-4-amine (Compound No. 35),

m/z: (M⁺+1) 438.34

¹HNMR (δ, CDCl₃): 7.98 (1H, s), 6.39-6.41 (1H, d), 4.48-4.54 (2H, m), 4.09-4.13 (2H, m), 3.99-4.03 (2H, m), 3.58-3.64 (2H, m), 3.15 (2H, s), 2.11-2.18 (4H, m), 1.87-1.91 (2H, m), 1.63-1.81 (6H, m), 1.49-1.53 (3H, t).

1-Ethyl-5-(1-oxa-2-azaspiro[4.5]dec-2-en-3-yl)-N-(tetrahydro-2H-pyran-4-yl)-6-(trifluoromethyl)-1H-pyrazolo[3,4-b]pyridin-4-amine (Compound No. 36),

m/z: (M⁺+1) 452.39

¹HNMR (δ, CDCl₃): 7.98 (1H, s), 6.33-6.35 (1H, d), 4.47-4.54 (2H, q), 4.00-4.11 (3H, m), 3.56-3.66 (2H, m), 2.96 (2H, s), 2.10-2.18 (2H, m), 1.82-1.84 (2H, m), 1.50-1.73 (13H, m).

1-Ethyl-N-(tetrahydro-2H-pyran-4-yl)-6-(trifluoromethyl)-5-(1,9,12-trioxa-2-azadispiro[4.2.4.2]tetradec-2-en-3-yl)-1H-pyrazolo[3,4-b]pyridin-4-amine (Compound No. 37),

m/z: (M⁺+1) 510.34

¹HNMR (δ, CDCl₃): 7.98 (1H, s), 6.33-6.34 (1H, d), 4.49-4.54 (2H, q), 4.13-4.15 (111, m), 3.97-4.00 (6H, m), 3.58-3.63 (2H, m), 3.00 (2H, s), 1.90-2.10 (8H, m), 1.68-1.72 (4H, m), 1.49-1.53 (3H, t).

tert-butyl 3-[1-ethyl-4-(tetrahydro-2H-pyran-4-ylamino)-6-(trifluoromethyl)-1H-pyrazolo[3,4-b]pyridin-5-yl]-1-oxa-2,8-diazaspiro[4.5]dec-2-ene-8-carboxylate

(Compound No. 40),

m/z: (M⁺+1) 553.44

¹HNMR (δ, CDCl₃): 7.99 (1H, s), 6.38-6.39 (1H, d), 4.50-4.54 (2H, q), 4.10-4.15 (1H, m), 3.99-4.04 (2H, m), 3.72-3.84 (2H, m), 3.58-3.64 (2H, m), 3.44 -3.48 (2H, m), 3.00 (2H, s), 2.11-2.14 (2H, m), 1.92-1.96 (2H, m), 1.63-1.69 (4H, m), 1.51-1.54 (3H, t), 1.48 (9H, s).

### Example 10 (a)

### Preparation of 4-Methylene Cyclohexanecarboxylic Acid Ethyl Ester

Potassium tert-butoxide (24 gm, 0.22 moles) and triphenylphosphine methyl iodide (78 gm, 28.22 moles) were dissolved in dry tetrahydrofuran (150 ml). The mixture was cooled to −78° C. and stirred at the same temperature for about 15 minutes. Ethyl 4-oxocyclohexane carboxylate (25 gm, 0.147 moles) in tetrahydrofuran was added drop wise and the mixture was stirred at the same temperature for about 30 minutes and then it was warmed to room temperature and stirred overnight, extraction was done with ethyl acetate and water. The organic layer was dried over sodium sulfate and concentrated. Purification was done by column chromatography using 5% ethyl acetate in hexane.

Yield: 19 gm (67%); m/z: (M⁺+1) 169.26

NMR:(δ, CDCl₃): 4.64 (2H, s), 4.09-4.15 (2H, q), 2.42- 2.47 (1H, m), 2.31- 2.39 (2H, m), 1.96-2.09 (414, m), 1.43- 1.62 (2H, m), 1.23- 1.26 (3H, t)

The following compounds were prepared similarly

{[(3-Methylidenecyclobutyl)methoxy]methyl}benzene

NMR: (δ, CDCl₃): 7.25-7.31 (5H, m), 4.74-4.76 (211, m), 4.53 (2H, s), 3.48-3.51 (2H, m), 2.78-2.82 (2H, m), 2.55-2.62 (1H, m), 2.40-2.45 (2H, m)

8-Methylidene-1,4-dioxaspiro [4.5]decane

NMR: (δ, CDCl₃): 4.66 (2H, s), 3.95 (4H, s), 2.29-2.26 (4H, t), 1.71-1.67 (4H, t)

tert-Butyl 4-methylidenepiperidine-1-carboxylate

NMR: (δ, CDCl₃) : 4.74 (2H, s), 3.41-3.42 (4H, d), 2.16- 2.18 (4H, d), 1.41 (9H, s)

### Example 11

### Preparation of Ethyl (cis*)-3-[4(cyclohexylamino)-1-ethyl-6-methyl-1H-pyrazolo[3,4-b]pyridin-5-yl]-1-oxa-2-azaspiro[4.5]dec-2-ene-8-carboxylate (Compound No. 12) and Ethyl (trans*)-3-[4-(cyclohexylamino)-1-ethyl-6-methyl-1H-pyrazolo [3,4-b ]pyridin-5 -yl]-1-oxa-2-azaspiro[4.5]dec-2-ene-8-carboxylate (Compound No. 13)

4-Methylene cyclohexanecarboxylic acid ethyl ester (2.2 gm, 0.0120 moles) (example 10 (a)) was added to 4-(cyclohexylamino)-1-ethyl-6-methyl-1H-pyrazolo[3,4-b]pyridine-5-carbaldehyde oxime (2 gm, 0.00632 moles) (example 9) in tetrahydrofuran (30 ml). The reaction mixture was stirred at room temperature for about 5 minutes. Sodium hypochlorite (20 ml) was added slowly to the reaction mixture over a period of about 5 minutes and the mixture was allowed to stir at room temperature for about 14 hrs. The organic solvent was evaporated and the residue was extracted in chloroform. The organic layer was concentrated and isomers were separated by column chromatography using 40% ethyl acetate in hexane.

Ethyl (cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylate (Compound No. 12),

Yield: 300 mg (9.67%); m/z: (M⁺+1) 468.15; HPLC purity : 98.14%

¹HNMR (6, CDCl₃): 7.89 (1H, s), 6.86 (1H, bs), 4.41-4.46 (2H, q), 4.13-4.18 (2H, q), 3.84 (1H, bs), 3.02 (2H, s), 2.51 (3H, s), 2.27-2.35 (1H, m), 2.02-2.07 (41-1, m), 1.95-1.99 (2H, m), 1.62-1.77 (2H, m), 1.58-1.60 (6H, m), 1.43-1.48 (3H, t), 1.25-1.29 (7H, m)

Ethyl (trans*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylate (Compound No. 13),

Yield: 500 mg (16.12%); m/z: (M⁺+1) 468.15; HPLC purity : 93.53%

¹HNMR: (δ, CDCl₃): 7.89 (1H, s), 6.73 (1H, bs), 4.41-4.46 (2H, q), 4.11-4.17 (2H, q), 3.84 (1H, bs), 3.06 (2H, s), 2.46 -2.5 (4H, m), 1.90- 2.08 (4H, m), 1.79-1.85 (4H, m), 1.58-1.67 (13H, m), 1.44-1.50 (3H, t)

The following compounds were prepared similarly

(Cis*)-7-[4-(cyclohexylamino)-1-ethyl-6-methyl-1H-pyrazolo[3,4-b]pyridin-5-yl]-5-oxa-6-azaspiro[3.4]oct-6-ene-2-carbonitrile (Compound No. 3),

m/z: (M⁺+1) 393.53; HPLC purity: 99.81% ¹HNMR: (δ, CDCl₃): 7.89 (1H, s), 6.99 (1H, bs), 4.41-4.47 (2H, q), 3.77 (1H, bs), 3.51 (2H, s), 3.20-3.22 (1H, m), 2.98-3.00 (2H, m), 2.95-2.97 (2H, m), 2.53 (311, s), 2.10-2.16 (2H, m), 1.80-2.08 (4H, m), 1.44-1.70 (6H, m), 1.42 (3H, t)

5-{(Cis*)-2-[(benzyloxy)methyl]-5-oxa-6-azaspiro [3.4]oct-6-en-7-yl}-N-cyclohexyl-1-ethyl-6-methyl-1H-pyrazolo[3,4-b]pyridin-4-amine (Compound No. 16),

m/z: (M⁺+1) 488.16; HPLC purity: 94.06%

¹HNMR: (6, CDCl₃): 7.88- 7.89 (1H, d), 7.33-7.34 (5H, m), 5.01 (2H, s), 4.43-4.53 (4H, q), 3.89 (1H, bs), 3.47 -3.48 (2H, m), 3.23 (2H, s), 2.10-2.27 (4H, m), 1.50-1.78 (5H, m), 1.25-1.49 (1H, m)

5-{(Trans*)-2-[(benzyloxy)methyl]-5-oxa-6-azaspiro[3.4]oct-6-en-7-yl}-N-cyclohexyl-1-ethyl-6-methyl-1H-pyrazolo[3,4-b]pyridin-4-amine (Compound No. 17),

m/z: (M⁺+1) 488.89; HPLC purity: 99.0%

¹HNMR: (δ, CDCl₃): 7.88 (1H, s), 7.29- 7.36 (51-1, m), 6.71 (1H, bs), 4.54 (2H, s), 4.41- 4.46 (211, q), 3.82 (1H, bs), 3.49 -3.53 (2H, m), 3.33 (2H, s), 2.50 (3H, s), 2.25-2.38 (5H, m), 1.49-1.78 (5H, m), 1.25-1.40 (8H, m)

Ethyl (Trans*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylate (Compound No. 21),

m/z: (M⁺+1) 470.91

¹HNMR (8, CDCl₃): 7.88 (1H, s), 6.99 (1H, s), 4.42-4.47 (2H, q), 4.12-4.17 (3H, m), 3.98-4.01 (2H, m), 3.57-3.62 (2H, m), 3.09 (2H, s), 2.53 (3H, s), 2.47 (1H, s), 2.10-2.12 (4H, m), 1.82-1.86 (4H, m), 1.26-1.60 (10H, m).

HPLC purity: 94%

Ethyl (cis*)-3-[1-ethyl-6-methyl-4-(tetrahydro-2H-pyran-4-ylamino)-1H-pyrazolo[3,4b]-pyridin-5-yl]-1-oxa-2-azaspiro[4.5]dec-2-ene-8-carboxylate (Compound No. 22), m/z: (M⁺+1) 470.91

¹HNMR: (δ, CDCl₃): 7.88 (1H, s), 7.08 (1H, s), 4.43-4.45 (2H, q), 4.13-4.18 (3H, m), 3.98-4.01 (2H, m), 3.60-3.62 (2H, m), 3.05 (2H , s), 2.53 (3H, s), 1.26-2.35 (18H, m).

HPLC purity: 85.39%

Ethyl (Trans*)- 3-[4-(cyclohexylamino)-1-ethyl-6-(trifluoromethyl)-1H-pyrazolo[3,4-b]pyridin-5 -yl]-1-oxa-2-azaspiro[4.5]dec-2-ene-8-carboxylate (Compound No. 38), m/z: (M⁺+1) 522.41

¹HNMR: (δ, CDCl₃): 7.98 (1H, s), 6.10-6.12 (1H, d), 4.47-4.53 (21-1, m), 4.12-4.17 (2H, m), 3.86-3.87 (1H, s), 2.98 (2H, s), 2.43-2.47 (1H, m), 2.09-2.11 (4H, m), 1.80-1.94 (14H, m), 1.45-1.49 (3H, m), 1.24-1.28 (3H, m).

Ethyl (cis*)- b 3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylate (Compound No. 39),

m/z: (M⁺+1) 522.48

¹HNMR: (δ, CDCl₃): 7.98 (1H, s), 6.27-6.28 (1H, d), 4.47-4.53 (2H, m), 4.13-4.19 (2H, m), 3.86 (1H, s), 2.95 (2H, s), 2.34-2.38 (1H, m), 1.79-2.17 (18H, m), 1.49-1.52 (3H, m), 1.26-1.29 (3H, t).

### Example 12

### Preparation of {(Cis*)-3-[4-(cyclohexylamino)-1-ethyl-6-methyl-1H-pyrazolo[3,4-b]pyridin-5-yl]-1-oxa-2-azaspiro[4.5]dec-2-en-8-yl}methanol (Compound No. 4)

Lithium aluminium hydride (12 mg, 0.00032 moles) was added portion wise under inert atmosphere to tetrahydrofuran (5 ml) cooled to 0° C. After about 15 minutes, solution of ethyl (cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylate (100 mg, 0.00024 moles) (example 11) in tetrahydrofuran (2 ml) was added drop wise at 0° C. The reaction mixture was stirred for about 3 hrs at room temperature. It was quenched by aqueous sodium sulfate, filtered through celite pad and the filtrate was extracted with ethyl acetate. The organic layer was dried over anhydrous sodium sulfate and concentrated in vacuo and the title compound obtained was purified by preparative thin layer chromatography using 80% ethyl acetate in hexane.

Yield: 80 mg (87.91%); m/z: (M⁺+1) 426.55; HPLC purity: 97.05%

¹HNMR: (δ, CDCl₃): 7.89 (1H, s), 6.76-6.78 (1H, d), 4.41-4.46 (2H, q), 3.84-3.86 (1H, bs), 3.54-3.55 (2H, d), 3.01 (2H, s), 2.51 (3H, s), 2.07-2.10 (4H, t), 1.77-1.79 (4H, t), 1.56-1.65 (10H, m), 1.50-1.54 (3H, t)

The following compound was prepared similarly

{(Trans*)-3-[4-(cyclohexylamino)-1-ethyl-6-methyl-1H-pyrazolo[3,4-b]pyridin-5-yl]-1-oxa-2-azaspiro[4.5]dec-2-en-8-yl}methanol (Compound No. 18),

m/z: (M⁺+1) 426.89 ; HPLC purity: 94.89%

¹HNMR: (δ, CDCl₃): 7.89 (1H, s), 6.66 (1H, s), 4.41-4.47 (2H, q), 3.70 (1H, bs), 3.51-3.53 (2H, d), 3.05 (2H, s), 2.51 (3H, s), 1.58- 2.17 (20H, m), 1.47-1.50 (3H, t)

### Example 13

### Preparation of N-cyclohexyl-1-ethyl-5-[(cis*)-8- fluoromethyl)-1-oxa-2-azaspiro[4.5]dec-2-en-3-yl]-6-methyl-1H-pyrazolo[3,4-b]pyridin-4-amine (Compound No.

{(Cis*)-3-[4-(cyclohexylamino)-1-ethyl-6-methyl-1H-pyrazolo [3,4-b]pyridin-5-y1]-1-oxa-2-azaspiro[4.5]dec-2-en-8-yl}methanol (25 mg, 0.00058 moles) (example 12) was dissolved in chloroform (5 ml). At 0° C., diethylamino sulphurtrifluoride (0.011 ml, 0.000088 moles) was added drop wise. The reaction mixture was stirred at 0° C. for about 10 minutes and then at room temperature for about 30 minutes. It was quenched by using 5% sodium carbonate solution. Extraction was done by using chloroform. The organic layer was dried and concentrated in vacuo. Crude product was purified by preparative thin layer chromatography using 50% ethyl acetate in hexane.

Yield: 10 mg (40%); m/z: (M⁺+1) 428.45; HPLC purity : 86.62%

¹HNMR: (δ, CDCl₃): 7.89 (1H, s), 6.80 (1H, bs), 4.41-4.46 (2H, q), 4.35-4.37 (1H, d), 4.23-4.25 (1H, d), 3.86 (1H, bs), 3.02 (2H, s), 2.08-2.10 (4H, m), 1.67-1.77 (14H, m), 1.56-1.59 (4H, m), 1.47-1.50 (3H, t).

### Example 14

### Preparation of (trans*)-3-[4-(cyclohexylamino)-1-ethyl-6-methyl-1H-pyrazolo[3,4-b]pyridin-5-yl]-1-oxa-2azaspiro[4.5]dec-2-ene-8-carboxylic acid (Compound No. 5)

Ethyl (trans*)-3-[4-(cyclohexylamino)-1-ethyl-6-methyl-1H-pyrazolo[3,4-b]pyridin- 5 -yl]-1-oxa-2-azaspiro[4.5]dec-2-ene-8-carboxylate (50 mg, 0.000107 moles) (example 11) was dissolved in ethanol (5 ml). Lithium hydroxide (22 mg, 0.00053 moles) in water (2 ml) was added and mixture was stirred at room temperature for about 14 hrs. The solvent was removed under vacuo, mixture was acidified with aqueous hydrochloric acid and extracted with ethyl acetate. The organic layer was dried and concentrated in vacuo to give final product.

Yield: 40 mg (79.78%); m/z: (M⁺+1) 440.52; HPLC purity: 98.13%

¹HNMR (δ, CDCl₃): 7.89 (1H, s), 6.77 (1H, bs), 4.41-4.47 (2H, q), 3.84 (1H, bs), 3.06 (2H, s), 2.55 (1H, bs), 2.51 (3H, s), 1.85-2.18 (19H, m), 1.46-1.50 (3H, t)

The following compounds were prepared similarly

(Cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylic acid (Compound No. 6),

m/z: (M⁺+1)440.75 ; HPLC purity: 99.26%

¹HNMR: (δ, CDCl₃): 7.90 (1H, s), 6.87 (1H, bs), 4.41-4.46 (2H, d), 3.86 (1H, bs), 3.03 (2H, s), 2.51 (3H, s), 2.39-2.44 (1H, m), 1.25-2.10 (22 H, m)

(Trans*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 24),

m/z: (M⁺+1) 442.83

¹HNMR: (δ, CDCl₃): 7.89 (1H, s), 7.08 (1H, bs), 4.43-4.48 (2H, q) 3.98-4.01 (3H, m), 3.58-3.64 (2H, m), 3.09 (2H, s), 2.54 (3H, s), 1.21-2.13 (16H, m).

HPLC purity: 91.41%

(Cis*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 25),

m/z: (M⁺+1) 442.76

¹HNMR: (δ, CDCl₃): 7.89 (1H, s), 7.09 (1H, s), 4.42-4.48 (2H, q), 3.99-4.09 (3H, m), 3.58-3.63 (2H, m), 3.45-3.49 (1H, m), 3.06 (2H, s), 2.54 (3H, s), 1.21-2.13 (15H, m).

HPLC purity: 91.71%

(Trans*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylic acid (Compound No. 32),

m/z: (M⁺+1) 494.34

¹HNMR: (d, CDCl₃): 7.94 (1H, s), 6.11 (1H, bs), 4.45-4.48 (2H, m), 3.82-3.85 (1H, m), 2.94 (211, s), 2.48-2.52 (2H, m), 2.08-2.13 (3H, m), 1.67-1.88 (12H, m), 1.42-1.46 (3H, m), 1.32-1.38 (2H, m).

HPLC purity: 99.38%

(Cis*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylic acid (Compound No. 33),

m/z: (M⁺+1) 494.34

¹HNMR: (δ, CDCl₃): 7.94 (1H, s), 6.22-6.24 (1H, d), 4.43-4.48 (2H, m), 3.80-3.85 (1H, m), 2.91 (2H, s), 2.33-2.39 (2H, m), 1.94-2.08 (10H, m), 1.74-1.77 (2H, m), 1.55-1.66 (4H, m), 1.46-1.48 (3H, m), 1.34-1.36 (2H, m).

HPLC purity: 98.40%

### Example 15

### Preparation of N-cyclohexyl-1-ethyl-6-methyl-5-[(cis*)-2-(1H-tetrazol-5-yl]-5-oxa-6-azaspiro[3.4]oct-6-en-7-yl-1H-pyrazolo[3,4-b]byridin-4-amine (Compound No.

(Cis*)-7-[4-(cyclohexylamino)-1-ethyl-6-methyl-1H-pyrazolo[3,4-b]pyridin-5-yl]-5-oxa-6-azaspiro[3.4]oct-6-ene-2-carbonitrile (30 mg, 0.000076 moles) (example 11) was dissolved in toluene (5 ml). Sodium azide (7.5 mg, 0.00011 moles) and triethyl amine hydrochloride (20 mg, 0.00015 moles) were added and reaction mixture was refluxed overnight. Toluene was removed under reduced pressure and reaction was neutralized by aqueous hydrochloric acid. Extraction was done by using chloroform, organic layer was dried and concentrated in vacuo. Crude product was purified by preparative thin layer chromatography using 50% ethyl acetate in hexane.

Yield: 30 mg (15.38%); m/z: (M⁺+1) 436.39; HPLC purity: 99.64%

¹HNMR: (δ, CDCl₃): 7.79 (1H, s), 7.13 (1H, bs), 4.33 (2H, bs), 3.67 (1H, bs), 3.18 (2H, s), 2.7 (2H, bs), 2.51 (2H, bs), 2.31 (3H, s), 2.00 (2H, bs), 1.68 (2H, bs), 1.33-1.39 (3H, q), 1.23-1.28 (8H, m).

### Example 16

### Preparation of N-cyclohexyl-1-ethyl-5-1[(cis*)-8-(methoxymethyl)-1-oxa-2-azaspiro[4.5]dec-2-en-3-yl]-6-methyl-1H-pyrazolo[3,4-b]pyridin-4-amine (Compound No.

{(Cis*)-3-[4-(cyclohexylamino)-1-ethyl-6-methyl-1H-pyrazolo[3,4-b]pyridin-5-yl]-1-oxa-2-azaspiro[4.5]dec-2-en-8-yl}methanol (30 mg, 0.0000705 moles) (example 12) in dimethylformamide (2 ml) was added to solution of sodium hydride (18 mg, 0.000077 moles) in dimethylformamide (5 ml) at 0° C. and the mixture was stirred for about 30 minutes. Methyl iodide (43 microlitre, 0.0000075 moles) was added dropwise at 0° C. The reaction mixture was stirred for about 3 hrs at room temperature. It was diluted with water and extracted with ethyl acetate. The organic layer was dried and concentrated in vacuo. Crude product was purified by preparative thin layer chromatography using ethyl acetate.

Yield: 12 mg (40%); m/z: (M⁺+1) 440.82; HPLC purity: 96.46.%

¹HNMR: (δ, CDCl₃): 8.02 (1H, s), 7.91 (1H, s), 4.45-4.51 (2H, q), 3.72-3.78 (1H, m), 3.64 (3H, s), 3.53-3.55 (2H, d), 3.36 (2H, s), 2.57 (3H, s), 2.08-2.09 (2H, m), 1.75-1.84 (414, m), 1.48-1.68 (12 H, m), 1.11-1.39 (4 H, m).

The following compound was prepared similarly

N-cyclohexyl-5-[(cis*)-8-(ethoxymethyl)-1-oxa-2-azaspiro[4.5]dec-2-en-3-yl]-1-ethyl-6-methyl-1H-pyrazolo[3,4-b]pyridin-4-amine (Compound No. 20)

m/z: (M⁺+1) 454.81 ; HPLC purity: 95.32%

¹HNMR: (8, CDCl₃): 7.94 (1H, s), 4.47-4.53 (2H, q), 3.64-3.91 (1H, m), 3.54-3.58 (2H, m), 2.96 (2H, s), 2.60 (3H, s), 1.86-2.10 (4H, m), 1.75-1.83 (12H, m), 1.62-1.67 (811, m), 1.55-1.58 (3H, t).

### Example 17

### Preparation of 3-[4-(cyclohexylamino)-1-ethyl-6-methyl-1H-pyrazolo[3,4b]-pyridin-5-yl]-1-oxa-2-azaspiro[4.5]dec-2-en-8-one (Compound No. 27)

N-cyclohexyl-1-ethyl-6-methyl-5-(1,9,12-trioxa-2-azadispiro[4.2.4.2]tetradec-2-en-3-yl)-1H-pyrazolo[3,4-b]pyridin-4-amine (120 mg, 0.00026moles) (example 10) was dissolved in dichloromethane (4 ml). Water (0.120 ml) was added to it followed by drop wise addition of trifluoroacetic acid (0.150 ml, 0.00132 moles) at 0° C. The reaction mixture was stirred at room temperature for about 6 hrs. Quenching was done by sodium bicarbonate solution. Extraction was done by ethyl acetate. Organic layer was dried over anhydrous sodium sulphate and concentrated in vacuo.

Yield: 80 mg (73.8%)

m/z: (M⁺+1) 409.52

¹HNMR: (δ, CDCl₃): 7.90 (1H, s), 6.94 (1H, s), 4.41-4.47 (2H, q), 3.86 (1H, bs), 3.18 (2H, s), 2.53 (3H, s), 2.34-2.42 (4H, m), 2.02-2.12 (4H, m), 1.25-1.78 (13H, m).

### Example 18

### Efficacy of Compounds

(a)(i) PDE4B Enzyme Assay

The efficacy of compounds as PDE4 inhibitors was determined by an enzyme assay using cell lysate of HEK293 cells transfected with PDE4B2 plasmids as PDE4B source. The enzyme reaction was carried out in the presence of cAMP (1 μM) at 30° C. in the presence or absence of test compound for 45-60 minutes. An aliquot of this reaction mixture was taken further for the ELISA assay and the protocol of the kit followed to determine level of cAMP in the sample. The concentration of the cAMP in the sample directly correlated with the degree of PDE4 enzyme inhibition. Results were expressed as percent control and the IC₅₀ values of test compounds were reported. IC₅₀ values of test compounds were found to be in the range of 5 nM to >1 μM.

(a)(ii) PDE7 Enzyme Assay

The efficacy of compounds as PDE7 inhibitors was determined by an enzyme assay using recombinant human PDE7A enzyme (I Med. Chem., 43, (2000), 683-689). The enzyme reaction was carried out in the presence of cAMP (1 μM) at 37° C. in the presence or absence of test compound for 60 minutes. An aliquot of this reaction mixture was taken further for the ELISA assay and the protocol of the kit was followed to determine level of cAMP in the sample. The concentration of the cAMP in the sample directly correlated with the degree of PDE7 enzyme inhibition. Results were expressed as percent control and the IC₅₀ values of test compounds, calculated using Graph pad prism, were found to be in the range of 55 nM to >1 μM.

(b) Cell Based Assay for TNF-α Release

Method of Isolation of Human Peripheral Blood Mononuclear Cells (PBMNC's)

Human whole blood was collected in vacutainer tubes containing heparin or EDTA as an anti coagulant. The blood was diluted (1:1) in sterile phosphate buffered saline and 10 ml was carefully layered over 5 ml Ficoll Hypaque gradient (density 1.077 g/ml) in a 15 ml conical centrifuge tube. The sample was centrifuged at 3000 rpm for 25 minutes in a swing-out rotor at room temperature. After centrifugation, interface of cells were collected, diluted at least 1:5 with PBS (phosphate buffered saline) and washed three times by centrifugation at 2500 rpm for 10 minutes at room temperature. The cells were resuspended in serum free RPMI 1640 medium at a concentration of 2 million cells/ml.

LPS (Lipopolysaccharide) Stimulation of Human PBMNC's

PBMN cells (0.1 ml; 2 million/ml) were co-incubated with 20 μl of compound (final DMSO concentration of 0.2%) for 10 minutes in a flat bottom 96 well microtiter plate. Compounds were dissolved in DMSO initially and diluted in medium for a final concentration of 0.2% DMSO. LPS (1 pg/ml, final concentration) was then added at a volume of 10 μl per well. After 30 minutes, 20 μl of fetal calf serum (final concentration of 10%) was added to each well. Cultures were incubated overnight at 37° C. in an atmosphere of 5% CO₂ and 95% air. Supernatant were then removed and tested by ELISA for TNF-a release using a commercial kit (e.g. BD Biosciences). The level of TNF-a in treated wells was compared with the vehicle (0.2% DMSO in RPMI medium) treated controls and inhibitory potency of compound was expressed as IC₅₀ values calculated by using Graph pad prism.

$$Percent inhibition = 100 - \frac{Percent TNF \text{-} α in drug treated}{Percent TNF \text{-} α in vehicle treated} \times 100$$

IC₅₀ values for some of the compounds were found to be in the range of ˜6 nM to 176 nM.

(c) In-Vitro Assay to Evaluate Efficacy of Compounds in Combination with p38 MAP Kinase Inhibitors or Corticosteroids

The assay is performed as described in (b) above, with individual compounds and their combinations tested at sub-optimal doses.

(d) In-Vitro Assay to Evaluate Efficacy of Compounds in Combination with β2- Agonists Measurement of Intracellular cAMP Elevation in U937 Cells

U937 cells (human promonocytic cell line) are grown in endotoxin-free RPMI 1640 with 25 mM HEPES (4-(2-hydroxyethyl)-1-piperazineethanesulfonic acid) medium containing 10% (v/v) heat-inactivated foetal bovine serum and 1% (v/v) of an antibiotic solution (5000 IU/ml penicillin, 5000 μg/ml streptomycin). Cells (0.25×10⁶/200 μl) are resuspended in Krebs' buffer solution and incubated at 37° C. for 15 minutes in the presence of test compounds or vehicle (0.2% DMSO in RPMI medium). The individual compounds and their combinations at sub-optimal doses are tested to evaluate synergistic or additive effect of the compounds. Generation of cAMP is initiated by adding 50 pl of 10 μM prostaglandin (PGE2) and the reaction stopped after 15 minutes, by adding 1N HCl (50 μl) and placed on ice for 30 minutes. The samples are centrifuged (450 g, 3 minutes), and levels of cAMP in the supernatant measured using cAMP enzyme-linked immunosorbent assay kit (Assay Designs). Percent inhibition is calculated by the following formula and IC₅₀ value determined using Graph pad prism.

$$Percent inhibition = 100 - \frac{Percent conversion in drug treated}{Percent conversion in vehicle treated} \times 100$$

We claim:

1. A compound having the structure of Formula I: 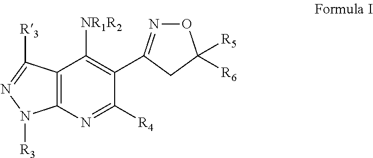
and its pharmaceutically acceptable salts, pharmaceutically acceptable solvates, stereoisomers, tautomers, racemates, regioisomers, geometric isomers, prodrugs, metabolites, polymorphs and N-oxides, wherein R₁ and R₂ independently are hydrogen, aryl, heteroaryl, —COR₇, —S(O)ₘR₇ (wherein R₇ is hydrogen, alkyl, cycloalkyl, aryl, 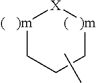
aralkyl, heteroaryl or heterocyclyl and m is an integer from 0-2), or (wherein m is an integer from 0-2 and X is —O—, S(O). (wherein m is an integer from 0-2), NR₈ {wherein R₈ is hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ (wherein R₇ and R′₇ are hydrogen, alkyl, cycloalkyl, aryl, aralkyl, heteroaryl or heterocyclyl and m is the same as defined above)}, C(═O), C═NOH or CR_{f}R_{q} (wherein R_{f} and R_{q} independently are hydrogen, halogen, hydroxy, cyano, NR₈R′₈ [wherein R₈ and R′₈ are hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ {wherein m, R₇ and R′₇ are the same as defined above}], —CONR₇R7, —COONR₇R₇′ or —COOR₇ (wherein R₇ and R′₇ are the same as defined above)),
R₃ is alkyl, aryl, cycloalkyl, heterocyclyl or heteroaryl,
R′₃ is hydrogen, alkyl, aryl, cycloalkyl, heteroaryl, heterocyclyl or (un) substituted amine,
R₄ is alkyl, aryl, cycloalkyl, halogen, cyano, heteroaryl, heterocyclyl, or (un) substituted amine,
R₅ and R₆ independently are alkyl, —CN, heterocyclyl, -(CH₂)—,C(═O)NRⱼR′ⱼ {wherein m is an integer from 0-2 and Rⱼ and R′ⱼ independently are hydrogen, alkyl, alkenyl, cycloalkyl, aryl, aralkyl, heterocyclyl, heteroaryl, heterocyclylalkyl, heteroarylalkyl or Rⱼ and R′ⱼ taken together with the nitrogen atom to which they are attached can form a optionally substituted heterocyclyl ring}, —(CH₂)ₘC(═O)ORⱼ {wherein m and Rⱼ are the same as defined above}, —(CH₂)ₘ₁ORⱼ {wherein m1 is an integer from 0-3 and Rⱼ is the same as defined above}or —(CH₂)ₘC(═O)heterocyclyl {wherein m is the same as defined above}, or R₅ and R₆ together can form a 3-7 membered saturated, partially saturated or unsaturated ring containing carbon atoms wherein one or more carbon atoms optionally can be replaced by heteroatoms selected from O, S(O)ₙ, {wherein m is an integer from 0-2}or NR₈ {wherein R₈ is the same as defined above}, or one or more carbon atoms optionally can be substituted with oxo, spino-attached heterocyclyl, cyano, alkyl, heteroaryl, heteroarylalkyl, —(CH₂)ₘhalogen, —(CH₂)ₘNR₇R′₇, —(CH₂)ₘOR₇, —(CH₂)ₘCONR₇R′₇, —(CH₂)ₘNR₇COR₇ or —(CH₂)ₘCOOR₇ (wherein m, R₇ and R′₇ are the same as defined above).

2. A compound according to claim 1 having the structure of Formula II: 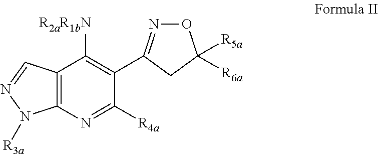
and its pharmaceutically acceptable salts, pharmaceutically acceptable solvates, stereoisomers, tautomers, racemates, regioisomers, geometric isomers, prodrugs, metabolites, polymorphs and N-oxides, wherein R_{1b} and R₂ₐ independently are hydrogen, 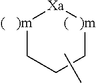
or (wherein m is an integer from 0-2 and Xa is —O— or —CH₂—),
R₃ₐ is alkyl,
R₄ₐ is alkyl, which may optionally be substituted with halogen,
R₅ₐ and Rₕₐ independently are —(CH₂)ₘ₁OH {wherein m1 is an integer from 0-3}or R₅ₐ and
R₆, together can form a 3-7 membered saturated, partially saturated or unsaturated ring containing carbon atoms wherein one or more carbon atoms optionally can be replaced by heteroatoms selected from O, S(O),, {wherein m is an integer from 0-2}or NR₈ {wherein R₈ is hydrogen, alkyl, alkenyl, alkynyl, cycloalkyl, cycloalkylalkyl, aryl, heteroaryl, heterocyclyl, —COR₇, —S(O)—,R₇, —COOR₇ or —CONR₇R′₇ (wherein R₇ and R′₇ are hydrogen, alkyl, cycloalkyl, aryl, aralkyl, heteroaryl or heterocyclyl and m is the same as defined above)}, or one or more carbon atoms optionally can be substituted with oxo, spiro-attached heterocyclyl, cyano, heteroaryl, heteroarylalkyl, —(CH₂)ₘhalogen, —(CH₂)ₘOR₇, or —(CH₂)ₘCOOR₇ (wherein R₇ and m are the same as defined above).

3. A compound, which is selected from
N-cyclohexyl-1-ethyl-6-methyl-5-[(cis*)-2-(1H-tetrazol-5-yl)-5-oxa-6-azaspiro[3.4]oct-6-en-7-yl]-1H-pyrazolo[3,4-b]pyridin-4-amine (Compound No. 1),
N-cyclohexyl-1-ethyl-5-[(cis*)-8-(fluoromethyl)-1-oxa-2-azaspiro[4.5]dec-2-en-3-yl]-6-methyl-1H-pyrazolo[3,4-b]pyridin-4-amine (Compound No. 2),
(Cis*)-7-[4-(cyclohexylamino)-1-ethyl-6-methyl-1H-pyrazolo[3,4-b]pyridin-5-yl]-5-oxa-6-azaspiro[3.4]oct-6-ene-2-carbonitrile (Compound No. 3),
{(Cis*)-3-[4-(cyclohexylamino)-1-ethyl-6-methyl-1H-pyrazolo[3,4-b]pyridin-5-yl]-1-oxa-2-azaspiro[4.5]dec-2-en-8-yl}methanol (Compound No. 4),
(Trans*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylic acid (Compound No. 5),
(Cis*)-3-[4-(cyclohexylamino)-1-ethyl-6-methyl-1H-pyrazolo[3,4-b]pyridin-5-yl]-1-oxa-2-azaspiro[4.5]dec-2-ene-8-carboxylic acid (Compound No. 6),
N-cyclohexyl-1-ethyl-6-methyl-5-(1-oxa-2-azaspiro[4.4]non-2-en-3-yl)-1H-pyrazolo[3,4-b]pyridin-4-amine (Compound No. 7),
1-Ethyl-6-methyl-5-(1-oxa-2-azaspiro[4.4]non-2-en-3-yl)-N-(tetrahydro-2H-pyran-4-yl)-1H-pyrazolo[3,4-b]pyridin-4-amine (Compound No. 8),
1-Ethyl-6-methyl-5-(1-oxa-2-azaspiro [4.5]dec-2-en-3-yl)-N-(tetrahydro-2H-pyran-4-yl)-1H-pyrazolo[3,4-b]pyridin-4-amine (Compound No. 9),
N-cyclohexyl-1-ethyl-6-methyl-5-(1-oxa-2-azaspiro[4.5]dec-2-en-3-yl)- 1H-pyrazolo[3,4-b]pyridin-4-amine (Compound No. 10),
1-Ethyl-6-methyl-5-(5-oxa-6-azaspiro[3.4]oct-6-en-7-yl)-N-(tetrahydro-2H-pyran-4-yl)-1H-pyrazolo[3,4-b]pyridin-4-amine (Compound No. 11),
Ethyl (cis*)-3-[4-(cyclohexylamino)-1-ethyl-6-methyl-1H-pyrazolo [3,4-b]pyridin-5-yl]-1-oxa-2-azaspiro[4.5]dec-2-ene-8-carboxylate (Compound No. 12),
Ethyl (trans*)-3-[4-(cyclohexylamino)-1-ethyl-6-methyl-1H-pyrazolo [3,4-b]pyridin-5-yl]-1-oxa-2-azaspiro[4.5]dec-2-ene-8-carboxylate (Compound No. 13),
{3-[4-(Cyclohexylamino)-1-ethyl-6-methyl-1H-pyrazolo [3,4-b]pyridin-5-yl]-4,5-dihydroisoxazole-5,5-diyl}dimethanol (Compound No. 14),
N-Cyclohexyl-1-ethyl-6-methyl-5-(5-oxa-6-azaspiro[3.4]oct-6-en-7-yl)-1H-pyrazolo[3,4-b]pyridin-4-amine (Compound No. 15),
5-{(Cis*)-2-[(benzyloxy)methyl]-5-oxa-6-azaspiro[3.4]oct-6-en-7-yl}-N-cyclohexyl-1-ethyl-6-methyl-1H-pyrazolo[3,4-b]pyridin-4-amine (Compound No. 16),
5-{(Trans*)-2-[(benzyloxy)methyl]-5-oxa-6-azaspiro[3.4]oct-6-en-7-yl}-N-cyclohexyl-1-ethyl-6-methyl-1H-pyrazolo[3,4-b]pyridin-4-amine (Compound No. 17),
{(Trans*)-3-[4-(cyclohexylamino)-1-ethyl-6-methyl-1H-pyrazolo[3,4-b]pyridin-5-yl]-1-oxa-2-azaspiro[4.5]dec-2-en-8-yl}methanol (Compound No. 18),
N-cyclohexyl-1-ethyl-5-[(cis*)-8-(methoxymethyl)-1-oxa-2-azaspiro[4.5]dec-2-en-3-yl]-6-methyl-1 H-pyrazolo[3,4-b]pyridin-4-amine (Compound No. 19),
N-cyclohexyl-5-[(cis*)-8-(ethoxymethyl)-1-oxa-2-azaspiro [4.5]dec-2-en-3 -yl]-1-ethyl-6-methyl-1H-pyrazolo[3,4-b]pyridin-4-amine (Compound No. 20),
Ethyl (trans*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylate (Compound No. 21),
Ethyl (cis*)-3-[1-ethyl-6-methyl-4-(tetrahydro-2H-pyran-4-ylamino)-1 H-pyrazolo[3,4-b]pyridin-5-yl]-1-oxa-2-azaspiro[4.5]dec-2-ene-8-carboxylate (Compound No. 22),
5-{2-[(Benzyloxy)methyl]-5-oxa-6-azaspiro[3.4]oct-6-en-7-yl}-1-ethyl-6-methyl-N-(tetrahydro-2H-pyran-4-yl)-1H-pyrazolo[3,4-b]pyridin-4-amine (Compound No. 23),
(Trans*)-3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 24),
(Cis*)- 3-[1-ethyl-6-methyl-4-(tetrahydro-2H-pyran-4-ylamino)-1H-pyrazolo[3,4-b]pyridin-5-yl]-1-oxa-2-azaspiro[4.5]dec-2-ene-8-carboxylic acid (Compound No. 25),
N-cyclohexyl-1-ethyl-6-methyl-5-(1,9,12-trioxa-2-azadispiro [4.2.4.2]tetradec-2-en-3-yl)-1H-pyrazolo[3,4-b]pyridin-4-amine (Compound No. 26),
3-[4-(Cyclohexylamino)-1-ethyl-6-methyl-1H-pyrazolo [3,4-b]pyridin-5-yl]-1-oxa-2-azaspiro[4.5]dec-2-en-8-one (Compound No. 27),
N-cyclohexyl-1-ethyl-5-(1-oxa-2-azaspiro [4.5]dec-2-en-3-yl)-6-(trifluoromethyl)-1H-pyrazolo[3,4-b]pyridin-4-amine (Compound No. 28),
N-cyclohexyl-1-ethyl-5-(1-oxa-2-azaspiro[4.4]non-2-en-3-yl)-6-(trifluoromethyl)-1H-pyrazolo[3,4-b]pyridin-4-amine (Compound No. 29),
N-cyclohexyl-1-ethyl-5-(5-oxa-6-azaspiro[3.4]oct-6-en-7-yl)-6-(trifluoromethyl)-1H-pyrazolo[3,4-b]pyridin-4-amine (Compound No. 30),
{3-[4-(Cyclohexylamino)-1-ethyl-6-(trifluoromethyl)-1H-pyrazolo[3,4-b]pyridin-5-yl]-4,5-dihydro-1,2-oxazole-5,5-diyl}dimethanol (Compound No. 31),
(Trans*)- 3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylic acid (Compound No. 32),
(Cis*)-3-[4-(cyclohexylamino)-1-ethyl-6-(trifluoromethyl)-1H-pyrazolo [3,4-b]pyridin-5-yl]-1-oxa-2-azaspiro[4.5]dec-2-ene-8-carboxylic acid (Compound No. 33),
1-Ethyl-5-(5-oxa-6-azaspiro[3.4]oct-6-en-7-yl)-N-(tetrahydro-2H-pyran-4-yl)-6-(trifluoromethyl)-1H-pyrazolo[3,4-b]pyridin-4-amine (Compound No. 34),
1-Ethyl-5-(1-oxa-2-azaspiro[4.4]non-2-en-3-yl)-N-(tetrahydro-2H-pyran-4-yl)-6-(trifluoromethyl)-1H-pyrazolo[3,4-b]pyridin-4-amine (Compound No. 35),
1-Ethyl-5-(1-oxa-2-azaspiro[4.5]dec-2-en-3-yl)-N-(tetrahydro-2H-pyran-4-yl)-6-(trifluoromethyl)-1H-pyrazolo[3,4-b]pyridin-4-amine (Compound No. 36),
1-Ethyl-N-(tetrahydro-2H-pyran-4-yl)-6-(trifluoromethyl)-5-(1,9,12-trioxa-2-azadispiro[4.2.4.2]tetradec-2-en-3-yl)-1H-pyrazolo[3,4-b]pyridin-4-amine (Compound No. 37),
Ethyl (trans*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylate (Compound No. 38),
Ethyl (cis*)-3-[4-(cyclohexylamino)-1-ethyl-6-(trifluoromethyl)-1H-pyrazolo[3,4-b]pyridin-5-yl]-1-oxa-2-azaspiro[4.5]dec-2-ene-8-carboxylate (Compound No. 39),
teat-butyl 3-[1-ethyl-4-(tetrahydro-2H-pyran-4-ylamino)-6-(trifluoromethyl)-1H-pyrazolo[3,4-b]pyridin-5 -yl]-1-oxa-2,8-diazaspiro [4.5]dec-2-ene-8-carboxylate (Compound No. 40),
and its pharmaceutically acceptable salts, pharmaceutically acceptable solvates, stereoisomers, tautomers, racemates, regioisomers, geometric isomers, prodrugs, metabolites, polymorphs and N-oxides.

4. A pharmaceutical composition comprising a therapeutically effective amount of a compound as defined in claim 1, 2 or 3 along with one or more of pharmaceutically acceptable carriers, excipients or diluents.

5. A pharmaceutical composition comprising a therapeutically effective amount of a compound of claim 1, 2 or 3, along with one or more of pharmaceutically acceptable carriers, excipients or diluents and at least one other compound selected from β2- agonists, corticosteroids, leukotriene antagonists, 5-lipoxygenase inhibitors, chemokine inhibitors, p38 kinase inhibitors, anticholinergics, antiallergics, PAF antagonists, EGFR kinase inhibitors, muscarinic receptor antagonists and combination(s) thereof.

6. A method for treating, preventing, inhibiting or suppressing inflammatory diseases, CNS diseases, pathological conditions and auto-immune disorders, in a mammal, comprising administering a therapeutically effective amount of a compound of claim 1, 2 or 3 or a therapeutically effective amount of a pharmaceutical composition of claim 4 or 5.

7. A method for the treatment, prevention, inhibition or suppression of multiple sclerosis, AIDS, rejection of transplant, rheumatoid arthritis, chronic obstructive pulmonary disease (COPD), asthma, bronchitis, allergic rhinitis, adult respiratory distress syndrome (ARDS), psoriasis, shock, atopic dermatitis, eosinophilic granuloma, allergic conjunctivitis, osteoarthritis, Crohn's disease, colitis, pancreatitis and cancer in a mammal comprising administering a therapeutically effective amount of a compound of claim 1, 2 or 3 or a therapeutically effective amount of a pharmaceutical composition of claim 4 or 5.

8. The method according to claim 6 or 7, wherein the disease is mediated through phosphodiesterase type 4 and/or 7.

9. A method for the preparation of a compound of Formula I, 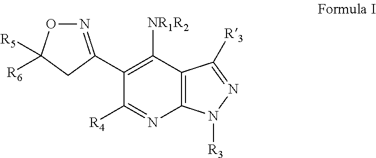
the method comprising,
(a) reacting a compound of Formula IV (wherein X is halogen and R₁ₐ is alkyl) with a compound of Formula V to give a compound of Formula VI, 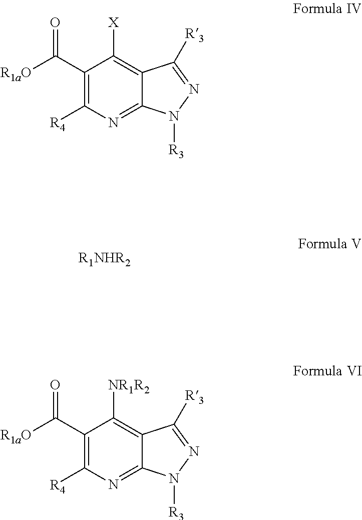
(b) carrying out ester hydrolysis of the compound of Formula VI to give a compound of Formula VII, 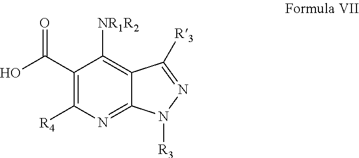
(c) reacting the compound of Formula VII with a compound of Formula VIII (wherein R₁ₐ is alkyl) to give a compound of Formula IX, 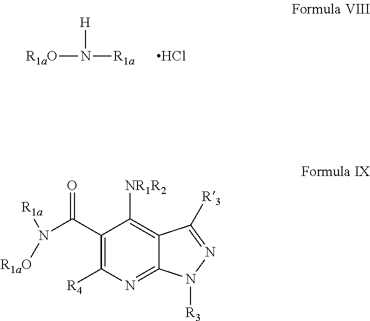
(d) carrying out reduction of the compound of Formula IX to give a compound of Formula X, 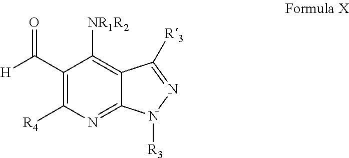
(e) reacting the compound of Formula X with hydroxylamine hydrochloride to give a compound of Formula XI, 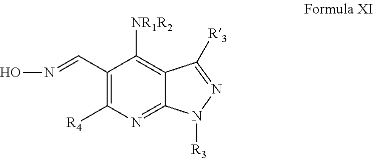
(f) reacting the compound of Formula XI with a compound of Formula XII 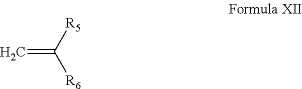
to give a compound of Formula I (wherein R₁, R₂, R₃, R′₃, R₄, R₅ and R₆ are the same as defined in claim 1).

10. A method for the preparation of a compound of Formula XIV, 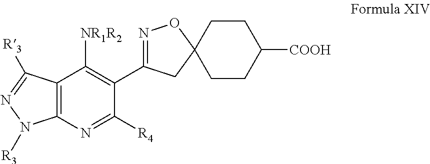
the method comprising carrying out ester hydrolysis of a compound of Formula XIII (wherein R₁ₐ is alkyl) 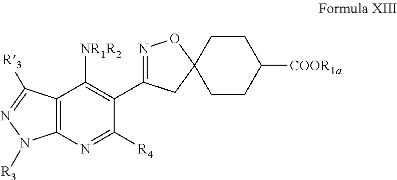
to give a compound of Formula XIV (wherein R₁, R₂, R₃, R′₃ and R₄ are the same as defined in claim 1).

11. A method for the preparation of a compound of Formula XV, 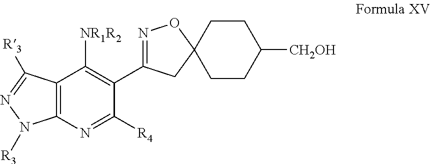
the method comprising carrying out reduction of a compound of Formula XIII (wherein R₁ₐ is alkyl) 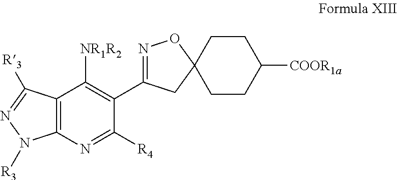
to give a compound of Formula XV (wherein R₁, R₂, R₃, R′₃ and R₄ are the same as defined in claim 1).

12. A method for the preparation of a compound of Formula XVI, 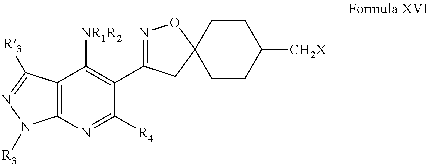
the method comprising halogenating a compound of Formula XV 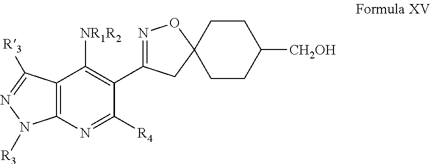
to give a compound of Formula XVI (wherein R₁, R₂, R₃, R′₃, R₄ are the same as defined in claim 1 and X is halogen).

13. A method for the preparation of a compound of Formula XVII, 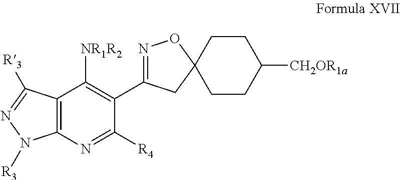
the method comprising reacting a compound of Formula XV 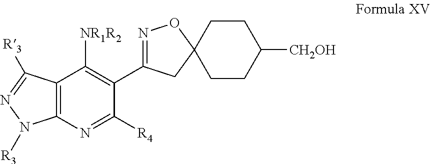
with a compound of Formula R₁ₐX (wherein X is halogen) to give a compound of Formula XVII (wherein R₁, R₂, R₃, R′₃, R₄ are the same as defined in claim 1 and R₁ₐis alkyl).

14. A method for the preparation of a compound of Formula XIX, 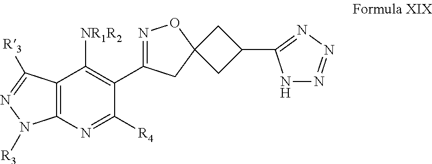
the method comprising cyclizing a compound of Formula XVIII 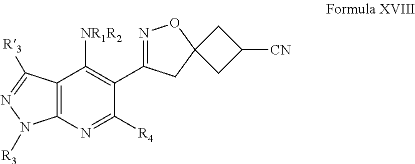
to give a compound of Formula XIX (wherein R₁, R₂, R₃, R′₃ and R₄ are the same as defined in claim 1).

15. A method for the preparation of a compound of Formula XXI, 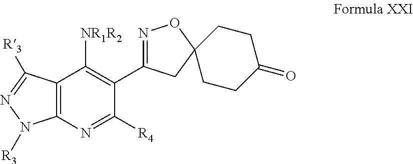
the method comprising carrying out hydrolysis of a compound of Formula XX 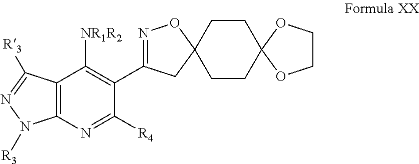
to give a compound of Formula XXI (wherein R₁, R₂, R₃, R′₃ and R₄ are the same as defined in claim 1).
